(19) United States
(12) Patent Application Publication
(10) Pub. No.: US 2006/0228707 A1
(43) Pub. Date: Oct. 12, 2006

(54) Eukaryotic genes involved in adult lifespan regulation

(75) Inventors: Cynthia Kenyon, San Francisco, CA (US); Jennifer Berman, San Francisco, CA (US); Andrew Dillin, San Diego, CA (US); Malene Hansen, San Francisco, CA (US); Ao-lin Hsu, Albany, CA (US); Coleen Murphy, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/536,635

(22) PCT Filed: Aug. 11, 2003
(86) PCT No.: PCT/US03/25266

§ 371 Date: May 26, 2005

Related U.S. Application Data
(60) Provisional application No. 60/402,213, filed on Aug. 9, 2002.
(60) Provisional application No. 60/413,988, filed on Sep. 26, 2002.
(60) Provisional application No. 60/482,993, filed on Jun. 27, 2003.

(51) Int. Cl. C12Q 1/68; C12Q 1/26; A01K 67/033; C40B 40/08

(52) U.S. Cl. 435/6; 435/25; 800/3; 800/13

(57) ABSTRACT

The present invention relates to regulation of lifespan in eukaryotes. More particularly, one aspect of the present invention is the identification of genes, gene products, and genes in pathways controlled by such genes and gene products, using RNAi and microarray analysis, that regulate lifespan (e.g., extend or truncate adult lifespan) in eukaryotes such as invertebrates (e.g., C. elegans), plants, and mammals, e.g., humans. The invention further relates to methods for identifying and using agents, including small molecule chemical compositions, antibodies, antisense nucleic acids, and ribozymes, that regulate, e.g., enhance, adult lifespan via modulation of aging associated proteins; as well as to the use of expression profiles, promoters, reporter genes, markers, and compositions in diagnosis and therapy related to lifespan extension, life expectancy, and aging. The present invention also relates to gene therapy involving lifespan associated genes.

### CROSS-REFERENCES TO RELATED APPLICATIONS

The present application claims priority to U.S. Ser. No. 60/402,213, filed Aug. 9, 2002, U.S. Ser. No. 60/413,988, filed Sep. 26, 2002, and U.S. Ser. No. 60/482,993, TTC Ref No. 02307O-119960, filed Jun. 27, 2003, each herein incorporated by reference in its entirety.

### STATEMENT AS TO RIGHTS TO INVENTIONS MADE UNDER FEDERALLY SPONSORED RESEARCH AND DEVELOPMENT

This invention was made with government support under Grant No. NIH AG11816, awarded by the NIH. The government has certain rights in this invention.

### FIELD OF THE INVENTION

The present invention relates to regulation of lifespan in eukaryotes. More particularly, one aspect of the present invention is the identification of genes, gene products, and genes in pathways controlled by such genes and gene products, using RNAi and microarray analysis, that regulate lifespan (e.g., extend or truncate adult lifespan) in eukaryotes such as invertebrates (e.g., C. elegans), plants, and mammals, e.g., humans. The invention further relates to methods for identifying and using agents, including small molecule chemical compositions, antibodies, antisense nucleic acids, double-stranded RNAs, small interfering RNAs, and ribozymes, that regulate, e.g., enhance, adult lifespan via modulation of aging associated proteins; as well as to the use of expression profiles, promoters, reporter genes, markers, and compositions in diagnosis and therapy related to lifespan extension, life expectancy, and aging. The present invention also relates to gene therapy involving lifespan associated genes.

### BACKGROUND OF THE INVENTION

Previously, classic genetic screens have been used to identify genes involved in the C. elegans development. In one example, inhibition of mitochondrial respiratory chain genes such as NADH ubiquinone oxidoreductase and ATP synthase in C. elegans larva was found to impair larval development and cause arrest in the third larval stage (see, e.g., Tsang et al., JBC 276:33240-33246 (2001)). In other examples, classical genetic screens have been used to identify C. elegans genes involved in a variety of processes, including dauer formation, and embryonic development. Some of these genes, for example the daf-2 and daf-16 genes, have been implicated in the regulation of lifespan see, e.g., Kenyon et al., Nature 366:461-464 (1993); Morris et al., Nature 382:536-539 (1996); Kimura et al., Science 277:942-946 (1997); Paradis et al., Genes Dev. 12:2488-2498 (1998); Paradis et al., Genes Dev. 13:1438-1452 (1999); Off & Ruvkun, Mol. Cell 2:886-893 (1998); Guarente & Kenyon, Nature 408:255-262 (2000); Ogg et al., Nature 389:994-999 (1997); and Lin et al., Science 278:1319-1322 (1997)).

Many different genes likely regulate the process of aging in eukaryotes and their identification will aid in understanding the process. Regulation of biological processes is frequently conserved between divergent organisms. For example, cell cycle proteins and their mechanism of action are conserved between organisms as divergent as yeast and humans. Thus, regulatory mechanisms identified in a genetically tractable organism can be used to predict and identify homologous and orthologous genes and gene products that regulate similar biological processes in higher eukaryotes. However, even in genetically tractable organisms, such as C. elegans, classical genetic methods are frequently labor intensive and cumbersome for identification of interesting mutants and for isolation of a gene of interest. The present invention solves these and other problems.

### SUMMARY OF THE INVENTION

The present invention provides a screen for identifying genes that interact with the reproductive system to regulate the aging of C. elegans. When the germ cell precursors of C. elegans are killed with a laser microbeam, lifespan is extended (see FIGS. 1-6). This extension requires the steroid hormone receptor DAF-12, the cytochrome P450 homolog DAF-9 and the forkhead-family transcription factor DAF-16. The longevity of these animals requires the presence of the somatic gonad (e.g., Z1 and Z4).

In another aspect, the present invention provides a temperature-sensitive mutation in the gene glp-1 that can be used to screen for genes that interact with the reproductive system to regulate aging in C. elegans. The mutant, (glp-1(e2141)) does not produce a germline when grown at high temperature, and as a result, this mutant lives longer than normal (Arantes-Oliveira et al., Science 295:502-505 (2001)). One strategy is to look for bacterial RNAi clones that prevent the lifespan extension of these animals but have only a small effect on wild-type lifespan (see FIGS. 1-6). This strategy can identify genes like daf-16, which are needed in order for germline-ablated animals to live longer than normal. Null mutations in this gene completely suppress the lifespan extension of glp-1 mutants, but have only a small (20%) reduction of wild-type lifespan.

In another aspect, the present invention provides genes required for the somatic gonad to extend lifespan. Eleven such genes were identified in a screen of Chromosome 1. The genes are shown in Table 1. These genes and gene products, regulatory sequences of these genes, and genes and gene products controlled by these genes are therefore useful, e.g., for developing drugs to regulate aging in eukaryotes, e.g., plants, mammals, humans.

In another aspect, the present invention provides data showing RNAi regulation of lifespan genes identified using either microarray analysis (Table 3) or a systematic RNAi screen of a bacterial feeding C. elegans RNAi library (Table 4). These genes and gene products, regulatory sequences of these genes, and genes and gene products controlled by these genes are therefore useful, e.g., for developing drugs to regulate aging in eukaryotes, e.g., plants, mammals, humans.

In another aspect, the present invention provides, DAF-16 regulated, age-associated genes and proteins identified using microarray and RNAi analysis (see Tables 5-7). Using DNA microarrays, genes were identified whose expression changed (over or underexpression) in C. elegans insulin/IGF-1 pathway mutants (long-lived (daf-2) or short-lived (daf-16 and daf-16; daf-2)). DNA microarrays were also used to identify genes whose expression changed in daf-2 or daf-16 RNAi treated animals. Functional analysis of the genes so identified was then carried out using RNAi. Such genes include cellular stress-response genes, antimicrobial genes, and metabolic genes, genes involved in synthesis of a steroid or lipid-soluble hormone. The gene ins-7 was also identified. These genes and gene products, regulatory sequences of these genes, and genes and gene products controlled by these genes are therefore useful, e.g., for developing drugs to regulate aging in eukaryotes, e.g., plants, mammals, humans. In addition, these genes can be used, e.g., as markers for the insulin/IGF system activity, as markers for the aging process, and as markers that indicate the likely longevity of an individual. The genes identified herein include any mammalian or human homologs and orthologs thereof. Certain genes may also be coordinately regulated by heat shock factor and by DAF-16, e.g., as identified using nucleic acid arrays in which HSF activity is impaired relative to wildtype or a daf-2 mutant strain in which HSF activity is impaired.

In one aspect, the present invention provides a method for identifying a compound that modulates adult aging, the method comprising the steps of: (i) contacting the compound with protein encoded by a gene listed in Tables 1 or 3-7; and (ii) determining the functional effect, e.g., lifespan effect or another age-associated parameter of the compound upon the polypeptide.

In one embodiment, the human homolog or ortholog is a human cellular stress-response gene, a human antimicrobial gene, a human metabolic gene, a human steroid or lipid-soluble hormone synthesis gene, or a human fatty acid desaturation gene.

In one embodiment, the human homolog or ortholog is a cytochrome P450, an estradiol-17-β-dehydrogenase, a alcohol/short-chain dehydrogenase, an esterase, a UDP-glucuronosyltransferase, an aminopeptidase, a carboxypeptidase, an amino-oxidase, an aminoacylase, an oligopeptide transporter, metallothionein, a receptor guanylate cyclase, a mitochondrial superoxide dismutase, a catalase, lysosyme, saposin, vitellogenin, glutathione-S-transferase, heat-shock protein, an F-box/cullin/Skp protein, an isocitrate lyase, a malate synthase ASMTL, insulin, IGF1 or IGF2.

In one embodiment, the functional effect is a physical effect or a chemical effect. In another embodiment, the functional effect is a phenotypic effect. In one embodiment, the polypeptide is expressed in a eukaryotic host or host cell, e.g., C. elegans. In another embodiment, the functional effect is determined by measuring longevity, average lifespan, or mean lifespan of an organism contacted with a compound. In one embodiment, the functional effect is determined by measuring enzymatic activity. In one embodiment, the functional effect is determined by measuring transcriptional activation. In one embodiment, the organism is C. elegans. In another embodiment, the organism is mammalian host or cell, e.g., a mouse, a rat, a guinea pig, a monkey, or a human.

In another embodiment, compounds that modulate aging are identified using computer programs that model age-associated protein structure and determining compounds that bind or interact with the modeled protein. Optionally, the effect of the compound can be validated by examining its effect on a cell or organism expressing the modeled age-associated protein.

In another embodiment, the method comprises providing a sequence comprising an age-associated protein, altering the sequence, e.g., by mutagenesis, and assaying the protein encoded by the altered sequence.

In another aspect, the present invention provides a method of modulating lifespan in a subject, the method comprising the step of contacting the subject with an therapeutically effective amount of a compound identified using the methods described herein. In one embodiment, the subject is C. elegans. In another embodiment, the subject is a mammalian subject, e.g., a mouse, a rat, a guinea pig, a monkey, or a human. In one embodiment, the subject is a plant.

In another aspect, the present invention provides a method of detecting the presence of a lifespan associated protein described herein, and the genes encoding such proteins in eukaryotic tissue, the method comprising the steps of: (i) isolating a biological sample; (ii) contacting the biological sample with a specific reagent that selectively associates with the protein of choice; and, (iii) detecting the level of specific reagent that selectively associates with the sample.

In one embodiment, the specific reagent is selected from the group consisting of: antibodies, oligonucleotide primers, and nucleic acid probes.

In another embodiment, the invention provides heterologous constructs comprising an age-associated gene as described herein or a promoter thereof, and a heterologous sequence such as a regulatory region, a reporter gene, a purification tag, e.g., for production of a fusion protein, for purification of a gene product, for more efficient expression of the gene or gene product, or for regulated expression of the gene or for expression of a reporter using the gene promoter.

In one embodiment, methods known to those of skill in the art such as RT-PCR, northern, Southern analysis, cDNA and genomic library cloning, etc. can be used to identify eukaryotic orthologs, e.g., invertebrate, vertebrate, plant, mammalian, and human orthologs, of the age-associated proteins provided herein. In another embodiment, computer sequence analysis can be used to identify orthologs. Such methods optionally include the step of assessing an age associated parameter in a cell in which the suspected ortholog is perturbed.

In one embodiment, endogenous or recombinant gene products of the age associated genes described herein are purified using the methods described herein, to at least about 50% purity, preferably 60%, 70%, 80%, 90% or higher purity. In another embodiment, the present invention provides a reaction mixture comprising an age-associated protein and another component such as a test compound, an antibody, a peptide, etc.

In another aspect, the invention features a nucleic acid that includes a regulatory sequence (e.g., a taanscriptional regulatory sequence) of a gene listed in Tables 1 or 3-7 or a homolog or ortholog thereof (e.g., a human or other mammalian homolog, as listed below) operably linked to a sequence encoding a detectable protein other than the protein encoded by the gene, e.g., a reporter protein, e.g., a protein that has an epitope tag, that can fluorescence, or that can catalyze a reaction. The invention also provides a transgenic organism that includes at least one cell that includes such a heterologous nucleic acid, and also organisms in which that cell that includes the heterologous nucleic acid also includes at least a second heterologous nucleic acid, e.g., a second reporter gene. The second heterologous nucleic acid can also include a regulatory sequence (e.g., a transcriptional regulatory sequence) of a gene listed in Tables 1 or 3-7 or a homolog or ortholog thereof (e.g., a human or other mammalian homolog) operably linked to a sequence encoding a detectable protein other than the protein encoded by the gene and other than the protein encoded by the first heterologous nucleic acid. In one embodiment, the two heterologous nucleic acids include regulatory sequences from different classes of genes (e.g., so that one heterologous nucleic acid includes a regulatory sequence from one class, and the other heterologous nucleic acid includes a regulatory sequence from another class). In another embodiment, they include regulatory sequences from the same class of genes. The regulatory sequence can be at least 100, 200, 500 bp, or at least 1, or 2 kb in length, e.g., between 0.1 and 5 kb, or 0.2 and 3 kb in length. The regulatory sequence can include at least one, two, three or four copies of the GTAAAt/cA motif and/or at least one, two, three or four copies of the CTTATCA motif. In one embodiment, the regulatory sequence includes a region from between about 10 bp 5′ of the ATG or first codon to about 400, 500, 700, or 900 bp 5′ of the ATG or first codon, or between about 100 bp 5′ of the ATG or first codon to about 1, 2, or 5 kb 5′ of the ATG or first codon. In one embodiment, the transgenic organism is an invertebrate, e.g., a nematode, e.g., C. elegans. For example, the C. elegans can include a mutation in one or more of daf-2, daf-16, daf-18, age-1, sir-2 or glp-1 or can be treated with an RNAi specific to such genes.

In one embodiment, a first and/or second reporter gene is used to categorize cells and or organisms (e.g., nematodes). The method can include separating cells according to category (e.g., sorting cells, e.g., using a flow cytometers (e.g., FACS). For example, the cells or organisms can include different mutations, be treated with different test compounds, or different nucleic acids (e.g., different plasmids).

In another aspect, the invention features a cell-free preparation that includes an isolated nucleic acid and a DNA-binding protein or a candidate DNA binding protein. The isolated nucleic acid includes a regulatory sequence of a gene listed in Tables 1 or 3-7 or a homolog or ortholog thereof (e.g., a human or other mammalian homolog). For example, the isolated nucleic acid is labeled. The isolated nucleic acid can be less than 900, 800, 500, 300, 200, or 120 nucleotides in length. Typically the nucleic acid is double stranded. The regulatory sequence can include at least one, two, three or four copies of the GTAAAt/cA motif and/or at least one, two, three or four copies of the CTTATCA motif. In one embodiment, the DNA-binding protein includes a DNA binding domain of daf-16 or another FOXO transcription factor (e.g., a mammalian FOXO TF). In one embodiment, the preparation includes at least two DNA binding proteins, for example, heat shock factor (HSF) and a FOXO TF. The preparation can further include a test compound, e.g., a non-protein test compound. The preparation can be used in a method for evaluating interaction between the protein and the isolated nucleic acid, e.g., to evaluate if a test compound alters the interaction between the DNA binding protein and the regulatory sequence. Methods for evaluating interactions with nucleic acid include fluorescence resonance, fluorescence polarization, electrophoretic mobility shift assays (MSA), and DNA footprinting. Analogous assays can also be performed in cells (e.g., using a one-hybrid assay). The method can further include contacting the test compound to a cell or organism and evaluating an age-associated parameter of the cell or organism.

The invention also provides a method of evaluating a test compound or a collection of test compounds. The test compound can be contacted to a cell or organism that includes a heterologous nucleic acid (e.g., reporter nucleic acid) described herein. An alteration in expression of the heterologous nucleic acid can indicate that the test compound affects lifespan regulation. The method can further include evaluating an age-related parameter of the cell or organism, e.g., if expression of the heterologous nucleic acid is altered. In one embodiment, cultured cells are used for the initial screening. Compounds from a collection that alter expression of the heterologous nucleic acid can then be contacted to an organism and an age-related parameter of the organism is evaluated. The organism can be from the same or a different species from the species of the cultured cell.

In another aspect, the invention features a method in which a test compound (e.g., from a collection of test compounds) is contacted to a cell or organisms. Expression of a plurality of genes in the cell or organism is evaluated. At least one of the genes is selected from Tables 1 or 3-7 or homologs or orthologs thereof. In one embodiment, at least 10, 20, 50, 80, or 100% of the genes evaluated are listed in Tables 1 or 3-7. For example, a first evaluated gene can be a class 1 gene of Table 5 and a second evaluated gene can be a class 2 gene of Table 6. In another example, at least 3, 4, 8, or 10 class 1 genes are evaluated, or at least at least 3, 4, 8, or 10 class 2 genes are evaluated. In one embodiment, fewer than 100, 50, 10 or 5 genes are evaluated in parallel (e.g., using a nucleic acid array). Similarly the expression level of proteins encoded by such genes or the post-translational state or sub-cellular localization of proteins encoded by such genes can be evaluated.

The invention also features a method of evaluating a subject. The method includes evaluating a parameter associated with a molecule (protein or nucleic acid) listed in Tables 1 or 3-7, or a homolog or ortholog thereof (e.g., a mammalian, e.g., human homolog) and recording the parameter (e.g., in a computer, e.g., a computer database) in association with information about the subject (e.g., an age-associated parameter). The method can including providing a risk factor or diagnosis (e.g., for aging or an age-associated process), or other information about lifespan regulation or an age-associated process in the subject. The evaluating can include evaluating mRNA or protein levels, enzymatic activity, or a post-translational modification. In one embodiment, the subject is a human, e.g., a human that has, is at risk for, or is being evaluated for, an age-related disorder.

In one embodiment, the subject is a post-natal subject, e.g., a subject that has not reached sexual maturity, or an adult, or a post-reproductive adult. For example, the subject can be a human that has not reached 10, 20, or 50 years of age. Or the subject can be a human that has attained at least 20, 40, 50, 70, 80, or 90 years of age.

The invention also features a method of evaluating at least one nucleotide in a genetic locus that includes a gene listed in Tables 1 or 3-7, or a homolog or ortholog thereof (e.g., a mammalian, e.g., human homolog). The method can include recording information about the at least one nucleotide in association with information about an age associated parameter. The method can be repeated for a plurality of organisms, e.g., a plurality of organism that are have a common or related age-associated parameter (e.g., characterized by at least 20, 30, 40% shorter or longer average expected lifespan). The information can be used for an association study, e.g., to evaluate a statistical or other association between a particular polymorphism and an age-associated parameter.

Some exemplary orthologs and homologs are listed as follows:

| | |
|---|---|
| gcy-6 | gi\|4505435\|ref\|NP_000897.1\|natriuretic peptide |
| | receptor A/guanylate cyclase A (atrionatriuretic peptide |
| | receptor A); Natriuretic peptide receptor |
| | A/guanylate cyclase A [Homo sapiens] |
| | gi\|113912\|sp\|P16066\|ANPA_HUMAN Atrial natriuretic |
| | peptide receptor A precursor (ANP-A) (ANPRA) (GC-A) |
| | (Guanylate cyclase) (NPR-A) (Atrial natriuretic peptide |
| | A-type receptor) |
| | gi\|68381\|pir∥OYHUAR natriuretic peptide receptor |
| | A precursor - human |
| | gi\|28230\|emb\|CAA33417.1\| ANP-A receptor preprotein |
| | (AA −32 to 1029) [Homo sapiens] |
| | gi\|3297986\|dbj\|BAA31199.1\| natriuretic peptide A type |
| | receptor [Homo sapiens] |
| | gi\|6013455\|gb\|AAF01340.1\|AF190631_1 natriuretic |
| | peptide receptor A [Homo sapiens] |
| gcy-18 | gi\|14349136\|emb\|CAC41350.1\| guanylate cyclase |
| | [Mus musculus] |
| | >gi\|728861\|sp\|P18293\|ANPA_MOUSE Atrial natriuretic |
| | peptide receptor A precursor (ANP-A) (ANPRA) (GC-A) |
| | (Guanylate cyclase) (NPR-A) (Atrial |
| | natriuretic peptide A-type receptor) |
| | gi\|2118323\|pir∥I57963 natriuretic peptide receptor |
| | A - mouse |
| | gi\|473634\|gb\|AAA66945.1\| natriuretic peptide receptor A |
| C54G4.6 | gi\|4757794\|ref\|NP_004183.1\| acetylserotonin |
| also dod- | O-methyltransferase-like; acetylserotonin |
| 18 | N-methyltransferase-like [Homo sapiens] |
| | gi\|3808148\|emb\|CAA75675.1\| ASMTL [Homo sapiens] |
| C46F4.2 | gi\|12669909\|ref\|NP_075266.1\| long-chain fatty-acid- |
| dod-9 | Coenzymegi\|4758332\|ref\|NP_004449.1\| long-chain |
| | fatty-acid-Coenzyme |
| | gi\|19911070\|dbj\|BAB86900.1\| Acyl-CoA synthetase 4 |
| | [Homo sap . . . |
| | gi\|2960069\|emb\|CAA73314.1\| acyl-CoA synthetase-like |
| | protein |
| | gi\|27469830\|gb\|AAH41692.1\| fatty-acid-Coenzyme A |
| | ligase, lo |
| | gi\|4758330\|ref\|NP_004448.1\| long-chain |
| | fatty-acid-Coenzyme |
| | gi\|7706449\|ref\|NP_057318.1] fatty-acid-Coenzyme |
| | A ligase |
| F32A5.5 | gi\|22538420\|ref\|NP_536354.2\| aquaporin 10; small |
| (MIP | intestine . . . |
| family) | gi\|10280624\|ref\|NP_066190.1\| aquaporin 9 |
| | [Homo sapiens] |
| | gi\|4826645\|ref\|NP_004916.1\| aquaporin 3 |
| | [Homo sapiens] |
| | gi\|1362754\|pir∥A57119 aquaporin 3 - human |
| | gi\|4502187\|ref\|NP_001161.1\| aquaporin 7; |
| | aquaporin adipose |
| | gi\|20137410\|sp\|Q96PS8\|AQPA_HUMAN Aquaporin 10 |
| | (Small intest . . . |
| | gi\|21912983\|dbj\|BAC05693.1\| aquaporin adipose |
| | [Homo sapiens |
| | gi\|17384411\|emb\|CAD13298.1\| bA251O17.3 (similar to |
| | aquapori |
| | gi\|25815123\|emb\|CAD38526.1\| aquaporin-3 |
| | [Homo sapiens |
| | gi\|18490903\|gb\|AAH22486.1\| aquaporin 1 |
| | (channel-forming int . . . |
| | gi\|2119185\|pir∥I52366 uterine water channel - human |
| | gi\|4502177\|ref\|NP_000376.1\| aquaporin 1; |
| | aquaporin 1 (chann . . . |
| | gi\|19387211\|gb\|AAL87136.1\| aquaporin 1 |
| | [Homo sapiens] |

1: NP_000404
hydroxysteroid (17-beta) dehydrogenase 1; Estradiol
17-beta-dehydrogenase-1 [Homo sapiens]
gi|4504501|ref|NP_000404.1|[4504501]
2: Q92506
Estradiol 17 beta-dehydrogenase 8 (17-beta-HSD 8)
(17-beta-hydroxysteroid dehydrogenase 8) (Ke6 protein) (Ke-6)
gi|12643402|sp|Q92506|DHB8_HUMAN[12643402]
3: P56937
Estradiol 17 beta-dehydrogenase 7 (17-beta-HSD 7)
(17-beta-hydroxysteroid dehydrogenase 7)
gi|8134404|sp|P56937|DHB7_HUMAN[8134404]
4: P51659
Estradiol 17 beta-dehydrogenase 4 (17-beta-HSD 4)
(17-beta-hydroxysteroid dehydrogenase 4)
gi|1706396|sp|P51659|DHB4_HUMAN[1706396]
5: P37059
Estradiol 17 beta-dehydrogenase 2 (17-beta-HSD 2) (Microsomal
17-beta-hydroxysteroid dehydrogenase) (20 alpha-hydroxysteroid
dehydrogenase) (20-alpha-HSD) (E2DH)
gi|544152|sp|P37059|DHB2_HUMAN[544152]
6: P14061
Estradiol 17 beta-dehydrogenase 1 (17-beta-HSD 1) (Placental
17-beta-hydroxysteroid dehydrogenase) (20 alpha-hydroxysteroid
dehydrogenase) (20-alpha-HSD) (E2DH)
gi|118554|sp|P14061|DHB1_HUMAN[118554]
7: S59136
estradiol 17beta-dehydrogenase (EC 1.1.1.62) type 4 - human
gi|2134658|pir∥S59136[2134658]
8: S43928
estradiol 17beta-dehydrogenase (EC 1.1.1.62) type 3 - human
gi|1085271|pir∥S43928[1085271]
9: A47287
estradiol 17beta-dehydrogenase (EC 1.1.1.62) type 2 - human
gi|539530|pir∥A47287[539530]
10: DEHUE7
estradiol 17beta-dehydrogenase (EC 1.1.1.62) type 1 [validated] -
human gi|65913|pir∥DEHUE7[65913]
11: NP_803882
cytochrome P450, family 20, subfamily A, polypeptide 1 isoform 1;
cytochrome P450 monooxygenase [Homo sapiens]
gi|29171730|ref|NP_803882.1|[29171730]
12: NP_065725
cytochrome P450, family 20, subfamily A, polypeptide 1 isoform 2;
cytochrome P450 monooxygenase [Homo sapiens]
gi|29171727|ref|NP_065725.2|[29171727]
13: NP_000932
P450 (cytochrome) oxidoreductase; Cytochrome P-450 reductase
[Homo sapiens]
gi|24307877|ref|NP_000932.1|[24307877]
14: NP_000766
cytochrome P450, family 2, subfamily J, polypeptide 2; cytochrome P450,
subfamily IIJ (arachidonic acid epoxygenase) polypeptide 2; microsomal
monooxygenase; flavoprotein-linked monooxygenase [Homo sapiens]
gi|18491008|ref|NP_000766.2|[18491008]
15: NP_476437
cytochrome P450, family 3, subfamily A, polypeptide 43 isoform 3;
cytochrome P450 polypeptide 43; cytochrome P450, subfamily IIIA,
polypeptide 43 [Homo sapiens]
gi|16933535|ref|NP_476437.1|[16933535]
16: NP_476436
cytochrome P450, family 3, subfamily A, polypeptide 43 isoform 2;
cytochrome P450 polypeptide 43; cytochrome P450, subfamily IIIA,
polypeptide 43 [Homo sapiens]
gi|16933533|ref|NP_476436.1|[16933533]
17: NP_000774
cytochrome P450, family 26, subfamily A, polypeptide 1 isoform 1;
cytochrome P450, subfamily XXVIA, polypeptide 1; P450, retinoic
acid-inactivating, 1; retinoic acid-metabolizing cytochrome;
retinoic acid 4-hydroxylase [Homo sapiens]
gi|16933530|ref|NP_000774.2|[16933530]
18: NP_476498
cytochrome P450, family 26, subfamily A, polypeptide 1 isoform 2;
cytochrome P450, subfamily XXVIA, polypeptide 1; P450, retinoic
acid-inactivating, 1; retinoic acid-metabolizing cytochrome;
retinoic acid 4-hydroxylase [Homo sapiens]
gi|16933528|ref|NP_476498.1|[16933528]
19: NP_061950
UDP glycosyltransferase 1 family, polypeptide A7;
UDP-glucuronosyltransferase 1A7 [Homo sapiens]
gi|29789084|ref|NP_061950.1|[29789084]
20: NP_061948
UDP glycosyltransferase 1 family, polypeptide A10;
UDP-glucuronosyltransferase 1A10 [Homo sapiens]
gi|29789078|ref|NP_061948.1|[29789078]

### BRIEF DESCRIPTION OF THE DRAWINGS

FIGS. 1-6 show lifespan screens and results using laser ablation of germ cell precursors or glp-1 (e2141ts) mutants.

FIGS. 7-10 show the effects of daf-2 and daf-16 on lifespan and lifespan data for select genes using RNAi analysis.

Tables 1 and 3-7 list genes involved in lifespan regulation.

Tables 2 and 8 provide RNAi regulation data for genes involved in lifespan regulation.

### DETAILED DESCRIPTION OF THE INVENTION

Introduction

Double-stranded RNA-mediated interference (RNAi) provides a sequence specific mechanism for inhibiting gene expression in C. elegans (see, e.g., Fire et al., Nature 391:806-811 (1998) and WO 99/32619)). This technique is also useful for functional genomics analysis of C. elegans genes (see, e.g., Fraser et al., Nature 408:325-330 (2000); Kamath et al., Genome Biol. 2:RESEARCH0002 (2000)).

Surprisingly, RNAi can be used to identify genes and gene products involved in lifespan and aging mechanisms in adults, by feeding bacteria expressing a dsRNA library over long-term time periods to C. elegans and measuring, e.g., mean and median lifespan increase or decrease in adults. Such assays can be used, in addition, to determine if two or more genes function together in a similar aging pathway, to determine if gene function is cell autonomous, and to determine if drug compounds known to alter the aging process function in the same or different pathways. Microarray assays can also be used to identify genes and gene products involved in lifespan and aging.

Microarray and RNAi assays are also useful in combination, e.g., by identifying a gene or gene product involved in lifespan using RNAi and then examining its expression pattern in old and young adults using microarray analysis, or by identifying a differentially expressed gene or gene product involved in lifespan using microarray analysis and then examining the effects of gene or gene product inhibition using RNAi. Microarray analysis can also be performed on animals treated with RNAi of genes identified using microarray analysis.

The present invention also provides screens for identifying genes that interact with the reproductive system to regulate the aging of C. elegans. When the germ cell precursors of C. elegans are killed with a laser microbeam, lifespan is extended. In another screen, the glp-1 mutant glp-1 e2141ts, which does not produce a germline when grown at high temperatures, is used to identify genes involved in aging. The effect of these genes on aging can be further analyzed using RNAi analysis.

The present invention provides gene and gene products shown in Tables 1 and 3-7, and homologs and orthologs thereof, e.g., mammalian and human homologs. The present invention also provides homologs and orthologs of these conserved proteins that are found in mammals, such as humans, and which are known to those of skill in the art. The invention therefore provides methods of screening for compounds, e.g., small molecules, antibodies, antisense molecules, and ribozyme, that are capable of modulating lifespan in adult eukaryotes, in particular, in mammals, e.g., for lifespan enhancement and treatment of premature aging.

In another aspect, the invention features a method that includes: contacting a test compound to a living or biochemical system that includes a target protein selected from those encoded by a gene listed in Tables 1 and 3-7, and homologs and orthologs thereof; and evaluating a property associated with the target protein or a direct substrate or binding partner thereof. For example, the method can include detecting the target protein (e.g., an interaction, conformation, chemical modification of the protein), a substrate, or binding partner (e.g., a binding protein). The method can be used, e.g., to evaluating a test compound, e.g., for an effect on lifespan or a lifespan-related process. In one embodiment, the target protein is a C. elegans protein. In another embodiment, the target protein is a mammalian protein.

The living or biochemical system can be a mammalian or non-mammalian system. In one embodiment, the system includes a cell extract, e.g., a lysate or fraction of a cell, e.g., a membrane preparation, a cytoplasmic preparation, or a partially or completely purified preparation. In another embodiment, the system includes isolated mitochondria, e.g., a cell extract or fraction enriched in mitochondria. In still another embodiment, the system includes a living cell, e.g., cultured cells, e.g., primary cell or transformed cells. In yet another embodiment, the system includes a living organism. The organism includes a cell that can express the target protein. In one embodiment, the target protein is heterologous to the system, e.g., it is expressed from an exogenously provided nucleic acid or the protein itself is provided exogenously, e.g., from a purified preparation.

The method can further include contacting the test compound to an organism (e.g., C. elegans, Drosophila, or a mammal, e.g., a mouse) and evaluating an age-associated parameter of the organism.

Exemplary properties include a catalytic parameter; structural conformation; post-translational modification; redox state; physical interaction of the target protein with another protein; metabolite formation or consumption; subcellular localization of the target protein; in vivo half-life of the target protein or target protein activity, transcription of a gene encoding the target protein or translation of the target protein.

In one embodiment, the catalytic parameter describes the catalytic properties of an enzyme, other than the target protein. For example, the target protein is a substrate of the enzyme. In one embodiment, the post-translational modification is a modification of the target protein. In another embodiment, the post-translational modification is catalyzed by the target protein. Exemplary post-translational modifications include phosphorylation, ubiquitination, methylation, acetylation/deacetylation, geranygeranylation, farnesylation, or proteolytic modification. In one embodiment, where the property relates to metabolite production or consumption, the metabolite can be a direct substrate or direct product of a reaction catalyzed or effected by the target protein. In another embodiment, where the property relates to metabolite production or consumption, the metabolite can be an indirect substrate or indirect product of a reaction catalyzed or effected by the target protein.

A culture cell used in the method can include a heterologous nucleic acid that encodes and expresses the target protein. The method can further include assessing whether the test compound directly interacts with the target protein.

In one embodiment, the target protein is operably linked to a reporter protein and the evaluating comprises evaluating the reporter protein.

In another aspect, the invention features a method that includes providing a nematode in which activity of a target protein is reduced in the organism by RNA interference; expressing a gene encoding a protein (e.g., candidate protein) that is heterologous to the organism; and evaluating an age associated parameter of the organism. The method can further include, prior to the evaluating, contacting the organism with a test compound. The candidate protein can be, for example, a mammalian protein. The method can include other features described herein.

In another aspect, the invention features a method that includes: providing a cell in which activity of a target protein is reduced in the organism by RNA interference; expressing a gene encoding a protein (e.g., candidate protein) that is heterologous to the organism; and evaluating an age associated parameter of the organism. The method can include other features described herein.

In still another aspect, the invention features a method that includes providing a cell or an organism (e.g., a nematode) in which activity of a target protein is reduced in the cell or one or more cells of the organism by RNA interference; contacting the organism or the cell with a test compound; and evaluating an age associated parameter of the organism.

In another aspect, the invention features a method that includes assessing an age-related parameter of a nematode that (1) expresses a heterologous gene in at least some cells; and (2) is deficient in at least some cells for an endogenous activity provided by a respiratory chain component. In one embodiment, the heterologous gene is from a non-nematode species, e.g., a mammalian species. In a related embodiment, the heterologous gene encodes a variant of a mammalian protein, the variant having between one and ten substitutions, insertions, or deletions. The heterologous gene can encode a domain of at least 30 amino acids from a mammalian protein or a variant thereof, having between one and six substitutions, insertions, or deletions.

The method can include contacting the organism with a test compound, e.g., prior to the assessing. The method can include other features described herein. For example, the method can include evaluating the organism, e.g., evaluating expression of one or more genes described herein.

In another aspect, the invention features a method of characterizing a protein, the method includes: providing a nucleic acid that encodes a protein having a subject amino acid sequence that contains at least one substitution, insertion, or deletion relative to a reference amino acid sequence, wherein the subject amino acid sequence and the reference amino acid sequence are at least 70% identical; expressing the nucleic acid in a culture cell or in an invertebrate cell; and evaluating an age-associated parameter of the cell, or an organism that includes the cell.

In another aspect, the invention features a C. elegans nematode that (1) expresses a heterologous gene in at least some cells, the heterologous gene encoding a heterologous protein (e.g., a protein described herein (e.g., a mammalian gene described herein) that is non-identical to a corresponding endogenous protein, or a functional domain thereof; and (2) is deficient in at least some cells for an endogenous activity provided by the corresponding endogenous protein.

In one embodiment, the heterologous gene encodes a mammalian protein. In another embodiment, the heterologous gene encodes a variant of a mammalian protein, the variant having between one and ten substitutions, insertions, or deletions. For example, the heterologous gene encodes at least a domain of at least 30 amino acids from a mammalian protein or a variant thereof, having between one and six substitutions, insertions, or deletions. The deficiency can be mediated by dsRNA, e.g., by RNA interference.

The invention also features a method that includes assessing an age-associated parameter of a nematode described herein, e.g., a nematode described above. In one embodiment, the method further includes, prior to the assessing, contacting the nematode to a test compound.

In another aspect, the invention features a non-human organism (e.g., a C. elegans nematode) that (1) is deficient in at least some cells for an endogenous activity, the deficiency generated by dsRNA in the cells, and (2) has an average lifespan of at least 24, 26, or 28 days (e.g., in the N2 background) or an average lifespan of at least 25% greater than the average lifespan of an otherwise identical nematode not contacted with the dsRNA. The nematode is such that, absent the deficiency, the nematode has an average lifespan of less than 22, 20, 18, or 16 days. In one embodiment, the nematode has functional genes for dauer pathway and/or functional genes for clk-1, gro-1, and/or another gene described herein. For example, the nematode can be wild-type with respect to a laboratory standard, e.g., the N2 background or another background. The dsRNA may include a strand that is complementary to a nucleic acid encoding a protein described herein, and human homologs and orthologs.

In another aspect, the invention features a method that includes: providing a nematode described herein, e.g., a nematode described above; introducing a heterologous gene that encodes a polypeptide into the nematode; expressing the heterologous gene in the nematode or a progeny of the nematode under conditions wherein the polypeptide is produced; and monitoring an age-associated parameter of the nematode or the progeny of the nematode. The heterologous gene can be, e.g., a nematode gene or a mammalian gene.

In another aspect, the invention features a nucleic acid vector that includes (1) a coding sequence encoding an amino acid sequence corresponding to a gene listed in Tables 1 and 3-7, or functional segment thereof; and (2) one or more of the following: (a) a promoter that is operably linked and heterologous to the coding sequence, and (b) a second coding sequence encoding a reporter protein or protein tag, the second coding sequence being operably linked and heterologous to the coding sequence.

In another aspect, the invention features a nucleic acid vector that includes (1) a first expressible coding sequence encoding an amino acid sequence corresponding to a gene listed in Tables 1, 3, 4, 5, 6, or 7, or functional segment thereof; and (2) a second expressible coding sequence encoding an amino acid sequence corresponding to a gene listed in Tables 1, 3, 4, 5, 6, or 7 (e.g., the same table as the first expressible coding sequence). In one embodiment, the vector can be used to overproduce a plurality of genes that extend lifespan. In one embodiment, the vector can be used to produce dsRNAs that inhibit a plurality of genes that reduce lifespan. In still other embodiments, the vector can be modified to produce a cell or non-human organism that has reduced lifespan capacity. Such a cell or organism can be used (e.g., a sensitized system) to evaluate test compounds, e.g., to identify a test compound that increases lifespan capacity.

In another aspect, the invention features a method that includes altering glyoxylate metabolism in a mammalian subject. In one embodiment, the subject is a non-hibernating mammal, e.g., a primate or human. The subject can be a subject identified as being in need of altered lifespan regulation. The method can include, e.g., increasing expression of a glyoxylate cycle gene (e.g., an isocitrate/malate synthase) gene in at least one cell of the subject, e.g., by administering a nucleic acid encoding the gene or by administering an agent that causes increased expression.

The invention also features a method that includes: providing a cell, organism, or biochemical system that includes a subject protein described herein; contacting an antibody that binds to the subject protein or antigen-binding fragment thereof to the cell or the organism; and evaluating an age-associated parameter of the cell, organism, or biochemical system.

Definitions

The genes and gene products listed in Tables 1 and 3-7 include (A) eukaryotic and mammalian nucleic acids and polypeptide polymorphic variants, alleles, mutants, and interspecies homologs that: (1) have an amino acid sequence that has greater than about 50% amino acid sequence identity, preferably 60%, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by the C. elegans genes provided herein; (2) bind to antibodies, e.g., polyclonal antibodies, raised against an immunogen comprising an amino acid sequence of a gene product listed in Tables 1 and 3-7 provided herein, and conservatively modified variants thereof; (3) specifically hybridize under moderately stringent hybridization conditions to an anti-sense strand corresponding to a nucleic acid sequence encoding a gene listed in Tablesl and 3-7 as provided herein, and conservatively modified variants thereof; (4) have a nucleic acid sequence that has greater than about 50%, preferably greater than about 60%, 65%, 70%, 75%, 80%, 85%, 90%, preferably 91%, 92%, 93%, 94%, 95%, 96%, 97%, 98% or 99% or higher nucleotide sequence identity, preferably over a region of at least about 25, 50, 100, 200, 500, 1000, or more nucleotides, to a protein as provided herein; and/or (5) sequences that genetically complement the C. elegans loss of function of a gene listed in Tables 1 and 3-7, as provided herein, (B) functional domains thereof, and (C) orthologs that are known in the art, but are not limited to category (A). A polynucleotide or polypeptide sequence is typically from a eukaryote, e.g., an invertebrate, vertebrate, or plant, preferably a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. A description of the sequence of the C. elegans genome can be found in Science 282:2012-2018 (1998).

Orthologs are biopolymeric sequences (e.g., nucleic acid or polypeptide sequences) that are found in different species, yet have sequence similarity (e.g., at least 20% sequence identify within a functional protein domain), and perform similar functions. In one embodiment (e.g., wherein multiple homologs are present) an ortholog is most homology to a reference sequence relative to other available sequences. In one embodiment, orthologs can be assigned by comparing numerous sequences to identify the best match-up. See, e.g., Science 1997 Oct. 24;278(5338):631-7 and Nucleic Acids Res 2001 Jan. 1; 29(1):22-28 for some exemplary methods and resource for assigning orthologs based on complete genome coverage.

With respect to embodiments that include one or more genes, or homologs or orthologs thereof from Tables 1 and 3-7, in some implementations, certain genes are excluded from the table, e.g., superoxide dismutases (e.g,. SOD-3) and metallothionens (e.g., mtl-1)can be excluded from the table.

The definition explicitly includes the human or mammalian homologs and orthologs or counterparts of each C. elegans aging associated gene or protein described herein, e.g., the genes and gene products listed in Tables 1 and 3-7.

The phrase “functional effects” in the context of assays for testing compounds that modulate activity of aging associated genes and proteins includes the determination of a parameter that is indirectly (e.g., upstream or downstream biochemical or genetic effects) or directly under the influence of aging associated proteins, e.g., a chemical or phenotypic effect, such as the ability to increase or decrease lifespan (see, e.g., Kenyon et al., Nature 366:461-464 (1993); Hsin & Kenyon, Nature 399:362-366 (1999); Apfeld & Kenyon, Cell 95:199-210 (1998); and Lin et al., Nature Genet. 28:139-145 (2001)) or, e.g., a physical effect such as ligand, cofactor or substrate binding or inhibition of ligand, cofactor or substrate binding. A functional effect therefore includes ligand, cofactor and substrate binding activity, changes in gene expression and gene expression levels in cells; changes in post transcriptional modification of a protein, e.g., phosphorylation or glycosylation; reporter gene or marker expression; changes in abundance and cellular localization; enzymatic activity; cellular half life; redox state; and structural conformation, etc.; and age-associated parameters, i.e., characteristics of young or old cells or organisms such as stress resistance, lifespan, doubling time, telomere length, physiological characteristics, appearance, disease states, etc. “Functional effects” include in vitro, in vivo, and ex vivo activities. The functional effect can be measured in a host cell, organelle (e.g., isolated mitochondria), host cell membrane, isolated organelle membrane (e.g., isolated mitochondrial membrane), cellular extract, organelle extract (e.g., mitochondrial extract) or host organism.

By “determining the functional effect” is meant assaying for a compound that increases or decreases a parameter that is indirectly or directly under the influence of aging associated proteins or genes, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape); chromatographic; or solubility properties for the protein; measuring inducible markers or transcriptional activation of the protein; measuring binding activity or binding assays, e.g. binding to antibodies; measuring changes in ligand binding activity; measuring cellular proliferation or lifespan; measuring cell surface marker expression; measurement of changes in protein levels for associated sequences; measurement of RNA stability; phosphorylation or dephosphorylation; signal transduction, e.g., receptor-ligand interactions, second messenger concentrations (e.g., cAMP, IP3, or intracellular Ca²⁺); identification of downstream or reporter gene expression (CAT, luciferase, β-gal, GFP and the like), e.g., via chemiluminescence, fluorescence, colorimetric reactions, antibody binding, inducible markers, and ligand binding assays.

“Ligand” refers to a molecule that is specifically bound by a protein.

“Substrate” refers to a molecule that binds to an enzyme and is part of a specific chemical reaction catalyzed by the enzyme.

“Cofactor” refers to an additional component required for activity of an enzyme. (Leninger, Principles of Biochemistry (1984); Stryer, Biochemistry (1995)). A cofactor may be inorganic such as Fe, Cu, K, Ni, Mo, Se, Zn, Mn or Mg ions, or an organic molecule also known as a coenzyme. Coenzymes include flavin adenine dinucleotide (FAD), nicotinamide adenine dinucleotide (NAD), nicotinamide adenine dinucleotide phosphate (NADP), heme, coenzyme A, pyrodoxal phosphate, thiamine pyrophosphate, 5′-deoxyadenosylcobalamine, biocytin, tetrahydrofolate, retinal, and 1,25-dihydroxycholecalciferol. A co-factor can also include a protein subunit bound to the co-factor.

“Inhibitors”, “activators”, and “modulators” of aging associated genes and proteins are used to refer to activating, inhibitory, or modulating molecules identified using in vitro and in vivo assays of aging associated proteins and genes. Inhibitors are compounds that, e.g., bind to, partially or totally block activity, decrease, prevent, delay activation, inactivate, desensitize, or down regulate the activity or expression of aging associated proteins and genes, e.g., antagonists. “Activators” are compounds that increase, open, activate, facilitate, enhance activation, sensitize, agonize, or up regulate aging associated proteins. Inhibitors, activators, or modulators also include genetically modified versions of aging associated proteins and genes, e.g., versions with altered activity, as well as naturally occurring and synthetic ligands, antagonists, agonists, antibodies, antisense molecules, ribozymes, small chemical molecules and the like. Such assays for inhibitors and activators include, e.g., expressing aging associated proteins in vitro, in cells, or cell membranes, applying putative modulator compounds, and then determining the functional effects on activity, as described above.

Samples or assays comprising aging associated proteins and genes that are treated with a potential activator, inhibitor, or modulator are compared to control samples without the inhibitor, activator, or modulator to examine the extent of inhibition. Control samples (untreated with inhibitors) are assigned a relative protein or gene activity value of 100%. Inhibition of aging associated proteins or genes is achieved when the activity value relative to the control is about 80%, preferably 50%, more preferably 25-0%. Activation of aging associated proteins or genes is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents (e.g., a “compound” that is being evaluated) as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, etc., to be tested for the capacity to directly or indirectly modulation lymphocyte activation. Exemplary small organic molecules have a molecular weight less than abou 8000, 6000, 5000, 4000, 3000 or 2000 Daltons.

The test compound can be in the form of a library of test compounds, such as a combinatorial or randomized library that provides a sufficient range of diversity. Members of the library can be present in the same container (e.g., as a pool that can be deconvolved or in a tagged formed (e.g., using a chemical, electronic, or nucleic acid tag (e.g., a display library)) or in different containers (e.g., each individually in a separate container). Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, e.g., C. elegans, most preferably a mammal such as a primate e.g., chimpanzee or human; cow; dog; cat; a rodent, e.g., guinea pig, rat, mouse; or a rabbit.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 50% identity, preferably 55%, 60%, 65%, 70%, 75%, 80%, 85%, 90%, 91%, 92%, 93%, 94%, 95%, 96%, 97%, 98%, 99%, or higher identity over a specified region (e.g., the C. elegans proteins provided herein), when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation () of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. In one embodiment, a “protein” or “polypeptide” includes a plurality of subunit chains, e.g., the quaternary structure of the protein or polypeptide is multimeric (e.g., homo- or hetero-dimeric). Accordingly, a “protein” or “polypeptide“may be a complex of different subunit chains. In another embodiment, a “protein” or “polypeptide” refers to a single chain.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs and orthologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., extracellular domains, transmembrane domains, and cytoplasmic domains. Domains are portions of a polypeptide that form a compact unit of the polypeptide and are typically 15 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization.

Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to aging associated proteins, polymorphic variants, alleles, orthologs, and conservatively modified variants, or splice variants, or portions thereof, can be selected to obtain only those polyclonal antibodies that are specifically immunoreactive with aging associated proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

Assays for Genes and Gene Products That Regulate Aging

Genetic and other models can be used to identify mutants, phenotypes (mediated by mutants and by RNAi), genes, and gene products in the aging process, e.g., RNAi analysis; microarray analysis; chemical mutagenesis; mammalian complementation assays for age-associated proteins; yeast two hybrid assays, immunoprecipitation; alteration in age-associated reporter gene expression or localization (e.g., daf-2 or daf-16); overexpression, underexpression, or knockout of gene expression, etc. Suitable controls include organisms with altered lifespan, e.g., by mutation or RNAi. These assays can be used with eukaryotic organisms, cells, and organelles such as mitochondria. The genes and gene products associated with a mutation are then identified and used to analyze the aging process at a molecular level. Genes and gene products that regulate the aging process can be identified under normal aging conditions. Patterns of gene expression that correlate with normal or abnormal aging can also be used to identify genes associated with aging. The aging process has likely been conserved throughout evolution. Thus, genes and gene products that regulate the aging process in one species will be useful to identify similar or orthologous genes and gene products in divergent species.

A. Manifestations of the Aging Process

The most obvious disruption of the aging process is a change in lifespan of an individual. Lifespan can either be increased or decreased by a mutation in a gene that participates in the aging process or, as shown here, by another intervention, e.g., RNAi mediated silencing of such a gene. In addition, for all eukaryotic organisms other physical characteristics can be used to distinguish young individuals from older individuals. Thus, at an organismal level, a mutation that affects the aging process will usually affect the lifespan of an individual and may also affect other aging characteristics of that individual. Such manifestations of the aging process are known as “age-associated parameters,” e.g., indicia from Nomarsid analysis, stress resistance, appearance, physiological changes, disease states, loss of doubling capacity, changes in differentiated phenotype, indirect effects such as fusion protein expression and localization or posttranscriptional modification, etc., are described in more detail below.

Those of skill in the art will recognize that the aging process can also be manifested at an organismal level or at a cellular level. While a list of characteristics of aging is provided below, it is not exhaustive and other characteristics of the aging process may also be analyzed within the scope of the present invention.

Characteristics of aging can be distinguished at the organismal level and may be species specific. For example, characteristics of older human individuals include skin wrinkling, graying of the hair, baldness, cataracts, hypermelanosis, osteoporosis, cerebral cortical atrophy, lymphoid depletion, thymic atrophy, increased incidence of diabetes type I, atherosclerosis, cancer, and heart disease (Nehlin et al., Annals NY Acad. Sci., 980:176-179 (2000)). Other characteristics of mammalian aging include the following: weight loss; lordokyphosis (hunchback spine); absence of vigor; lymphoid atrophy; decreases in bone density, dermal thickness, and subcutaneous adipose tissue; decreased ability to tolerate stresses, such as wound healing, anesthesia, and response to hematopoietic precursor cell ablation; sparse hair; liver pathologies; atrophy of intestinal vilm; skin ulceration; amyloid deposits; and joint diseases (Tyner et al., Nature 415:45-53 (2002)).

Many diseases and disorders also are associated with aging or increased age. Exemplary age-related diseases and disorders include: cancer (e.g., breast cancer, colorectal cancer, CCL, CML, prostate cancer); skeletal muscle atrophy; adult-onset diabetes; diabetic nephropathy, neuropathy (e.g., sensory neuropathy, autonomic neuropathy, motor neuropathy, retinopathy); obesity; bone resorption; age-related macular degeneration, ALS,, Bell's Palsy, atherosclerosis, cardiac diseases (e.g., cardiac dysrhythmias, chronic congestive heart failure, ischemic stroke, coronary artery disease and cardiomyopathy), chronic renal failure, type 2 diabetes, ulceration, cataract, presbiopia, glomerulonephritis, Guillan-Barre syndrome, hemorrhagic stroke, short-term and long-term memory loss, rheumatoid arthritis, inflammatory bowel disease, neurodegenerative disorders (e.g., Alzheimer's, Huntington's, Parkinson's), multiple sclerosis, SLE, Crohn's disease, osteoarthritis, pneumonia, and urinary incontinence. In addition, many disorders associated with protein aggregation (e.g., polyglutamine aggregation, amyloid formation, etc) or protein misfolding can also be age-related. Symptoms and diagnosis of diseases are well known to medical practitioners. A compound identified by a method described herein can be used to ameliorate at least one symptom of such diseases and disorders. Similarly, one or more genes described herein can be used to evaluate a risk, association, or status of such diseases and disorders.

Careful observation reveals characteristics of the aging process in other eukaryotes, including invertebrates. For example, characteristics of aging in the model nematode C. elegans as observed by Nomarski analysis include slow movement, flaccidity, yolk accumulation, intestinal autofluorescence (lipofuscin), loss of ability to chew and expel (distended oral and anal cavities), necrotic cavities in tissue, curdled appearing tissue, and germ cell appearance (graininess, large, well separated nuclei, fewer nuclei, and cavities).

Characteristics of aging can also be observed in cultured cells and also in mitochondria. Note that many of these characteristics can also be observed in animals. Normal eukaryotic cells have a defined lifespan when taken from the organism grown in culture. These “primary” tissue culture cells are cells that have neither been immortalized nor acquired a transformed phenotype. The primary cells will divide a defined number of times in culture and then die (reviewed in Campisi, Exper. Geron. 36:6-7-618 (2001)). Cellular aging is also characterized by changes other than loss of doubling capability, e.g. changes in apoptotic death and changes in differentiated phenotypes (Id.). In some cases, cellular characteristics of aging can also be observed in immortalized or transformed cell lines. Aging cells also show stress resistance, e.g., free radical generation and H₂O₂ resistance. Age-related bio-markers, gene, and protein expression patterns may also be used to determine or measure aging.

Finally, aging can be assessed indirectly, by an aging related functional effects (phenotypic, physical, and chemical effects), e.g., gene expression (e.g., transcript abundance), protein abundance/localization/modification state, chromatin structure, signal transduction, second messenger levels, marker expression, phosphorylation, posttranscriptional modification, reporter gene expression, reporter or fusion protein localization, etc. Such effects can often be monitored when examining upstream or downstream genetic or biochemical pathways of an aging associated gene. Such effects can also be monitored using the aging associated gene.

In one embodiment, a test compound is contacted to one or more cells of an organism or one or more culture cells, and the one or more cells, or the entire organism is evaluated. In particular, a characteristic of aging (e.g., a direct observation or an aging-related functional effect) can be evaluated to determine the test compound has an affect on aging or an aging-related process such as stress resistance or metabolism.

B. Isolation of Genes Associated with Aging

Those of skill in the art will recognize that aging associated nucleic acids and proteins may be conserved in divergent species. Thus, the sequence of a nucleic acid or protein associated with aging in one species can be used to identify aging associated nucleic acids and proteins from other species, as well as genetic and biochemical pathways for the aging associated genes. For example, using methods described in this specification, aging associated genes identified in C. elegans can be used to identify aging associated genes or proteins in humans or other higher eukaryotes.

Isolation of Genes and Gene Products Associated With Aging Using Classical Genetic Methods.

Using classical genetic methods (random genomic mutagenesis), aging mutants are be generated by mutagenesis. The mutagenesis protocol will depend on the organism. For example, some eukaryotic organisms can be randomly mutagenized chemically by treatment with compounds like ethane methyl sulfonate (EMS) or can be mutagenized by exposure to UV or gamma irradiation. Preferably, these compounds would be used on organisms such as mammalian cells, yeast, C. elegans, Drosophila melanogaster, or zebrafish.

Mutants in the aging process will preferably be characterized by an increase or a decrease in lifespan (e.g., at least 10, 20, 40, 50, 70, 90, 100, 120, 150% greater than wild-type, or at least 10, 20, 30, 40, 50, or 60% less than wild-type). Mutants in the aging process will also preferably exhibit a temporal change in expression of an aging characteristic, including those listed above. For example, a mutant can show alteration in expression of a gene or gene product thereof listed in Table 1, 3, 4, 5, 6, or 7. In one embodiment, the expression is more similar to a daf-2 mutant than it is to wildtype.

Those of skill in the art will recognize that mutants can be generated in many ways depending on the organism and phenotypes studied. Typically, the mutagenesis process decreases, increases, or changes gene activity. Examples of such mutants include age-1, daf-2, and daf-16 in C. elegans.

Isolation of Genes and Gene Products Associated With Aging Using Gene Inactivation.

In another embodiment, aging mutants are made by inactivation of a gene of interest, using methods other than classical genetic mutagenesis methods. The gene of interest can be inactivated, e.g., using dsRNA inhibition, by using antisense technology, or can be inactivated by homologous recombination. The inactivation can take place in a multicellular organism or in cultured cells. For example, the p66 gene has been inactivated from mice using homologous recombination, creating a mouse with a longer lifespan than wildtype. Transgenic mice of interest which show lifespan increase include Ames dwarf mutant mice, p66(−/−) knockout mice, alpha MUPA and MGMT transgenic mice (see, e.g., Anisimov, Mech Aging Dev. 122:1221-1255 (2001); Lithgow & Andersen, Bioessays 22:410-413 (2000)).

dsRNA inhibition can also be used to screen a large number of genes for a phenotype. DNA fragments corresponding to predicted genes are cloned into a vector between two bacterial promoters in inverted orientation. The library is then transformed into a bacterial strain capable of expressing the DNA fragments. The transformed bacteria or the library DNA alone is then introduced into the experimental organism. If desired, inducible promoters can be used and expression of the inhibitory dsRNA can be induced during a particular time of development or under desired conditions.

A preferred embodiment uses a library whose members each include a DNA fragment from C. elegans. Each library member is transformed into E. coli and the E. coli fed to the worms. The DNA fragments are under the control of T7 promoters. The bacteria express a T7 polymerase that is inducible by IPTG, rendering expression of the inhibitory dsRNA inducible by IPTG.

Isolation of Genes and Gene Products Associated With Aging Using Overexpression.

In another embodiment, aging mutants are made by overexpressing a gene associated with aging, using methods other than classical genetic mutagenesis methods.. The gene associated with aging is cloned into a vector under the control of a promoter appropriate for the experimental system. The expression vector is then introduced into the experimental system. The overexpression can take place in either a multicellular organism or in cultured cells.

Isolation of Genes and Gene Products Associated With Aging Using Naturally Occurring Mutants.

Aging mutants can also occur naturally. Those of skill in the art will recognize that such mutants do exist and can be used in the present invention. For example, in humans, several premature aging syndromes have been characterized including Werner syndrome, Hutchinson-Guilford disease, Bloom's syndrome, Cockayne's syndrome, ataxia telangiectasia, and Down's syndrome. Where appropriate, cells from an individual afflicted with an aging syndrome can be studied, rather than the whole organism.

Isolation of Genes and Gene Products Associated With Aging Using Genetic or Biochemical Pathways Known to Regulate Aging.

Genetic analysis can also be used to delineate regulatory pathways and determine functional relationships between genes and gene products. In the case of a complex biological process such as aging, more than one regulatory pathway may regulate the aging process. Those of skill in the art will recognize that genetic analysis of mutants can be used to characterize regulatory pathways and determine relationships between genes. Of course, it also possible to use RNA interference to modulate gene activity in analyzing the regulatory pathways and relationships.

An example of genetic analysis of a regulatory pathway is found in C. elegans. The daf-2 gene encodes an insulin/IGF-1 receptor homolog. Mutations that lower the level of daf-2 result in animals that have enhanced lifespans. (For review see Guarente and Kenyon, Nature 408:255-262 (2000)). daf-16 encodes a forkhead transcription factor homolog that acts downstream of daf-2 and is required for daf-2 activity. daf-16 mutants have short lifespans. Newly isolated mutations can be analyzed for interaction with the daf2/daf16 pathway. In that way, genes and gene products can be assigned to a regulatory pathway.

In addition, genes that interact with the pathway can be identified by using an appropriate mutant screen. For example, the C. elegans protein DAF-16 is a transcriptional activator. A fusion protein between DAF-16 and green fluorescent protein (DAF-16/GFP) can be used to identify the cellular location of the protein. In wild-type animals the protein is localized throughout cells. In long-lived daf-2 mutants, DAF-16 is localized to the nucleus.

Those of skill in the art will recognize that the localization of DAF-16/GFP can be used to identify mutants that pertutb the daf2/daf16 pathway. Localization of DAF-16/GFP to the nucleus can be used to screen for drugs that enhance lifespan or mutations that enhance lifespan. A similar fusion using an end product of the pathway, superoxide dismutase (SOD-3), can be similarly used. Levels of fluorescence from SOD-3/GFP can be followed by microscopy. Those of skill in the art will recognize that expression of SOD-3/GFP can be used to screen for long-lived mutants.

Isolation of genes and gene products associated with aging using changes in expression levels.

Those of skill in the art will recognize that levels of messenger RNA can be measured during the aging process. For aging associated proteins, changes in mRNA levels can be detected either during normal aging process or when comparing an aging mutant to a wild-type individual. Changes in mRNA levels can be measured using techniques known to those of skill in the art, including microarrays, northern blots, and RT PCR.

Aging associated genes can be identified through the use of microarrays where changes in expression of mRNA levels under different conditions or at different times of development can be assayed. mRNA levels can also be analyzed in aging mutants to identify genes that are affected by increases or decreases in lifespan.

Microarrays are made by methods known to those of skill in the art, or are purchased. Gene expression profiles for the genes described herein can be generated and used for comparison to identify other age-associated genes. The profile can be generated using a microarray, or by other means. The profiles can be derived from animals, cells, mitochondria, or other suitable sources expressing the genes of interest, e.g., RNAi treated cells or animals. Such profiles can be stored as computer files and analyzed or compared to identify additional genes using algorithms known to those of skill in the art.

Moreover, a gene identified by any method, e.g., transcript or protein profiling, RNAi, or genetic mutation, can then by analyzed by one of the other methods. For example, the activity of a gene whose transcription is correlated with aging can altered using RNAi. Further, chromosomal deficiencies and genetic mutations can be identified in the gene of interest. These exemplary alterations can be used to evaluate the contribution of a gene or gene product to the aging phenotype. The functional relevance of genes so identified can be tested with mutants or RNAi.

Computer Assisted Methods

Yet another assay for compounds that modulate aging involves computer assisted drug design, in which a computer system is used to generate a three-dimensional structure of an aging associated protein based on the structural information encoded by the amino acid or nucleic acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind, e.g., ligands, substrates, cofactors, etc. These regions are then used to identify ligands that bind to the protein.

The three-dimensional structural model of the protein is generated by entering an aging associated protein amino acid sequences of at least 25, 50, 75 or 100 amino acid residues or corresponding nucleic acid sequences encoding an aging associated protein into the computer system. The amino acid sequence represents the primary sequence or subsequence of each of the proteins, which encodes the structural information of the protein. At least 25, 50, 75, or 100 residues of the amino acid sequence (or a nucleotide sequence encoding at least about 25, 50, 75 or 100 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the aging-associated protein is then generated by the interaction of the amino acid sequence and the computer system, using software known to those of skill in the art. The resulting three-dimensional computer model can then be saved on a computer readable substrate. For example, three-dimensional models of the structures of a number of proteins described here are known and can be used to model homologs, orthologs and interactions with other chemical compounds. See, e.g., Damberger et al. Protein Sci. 1994 October;3(10):1806-21; Harrison et al. Science. 1994 Jan. 14;263(5144):224-7; Lange et al. Proc Natl Acad Sci USA. 2002 Mar. 5;99(5):2800-5; Iwata, et al., Science. 1998 Jul. 3;281(5373):64-71; Gibbons et al., Nat Struct Biol. 2000November;7(11):1055-61; Faig et al., (2001) Structure (Camb). 2001 August;9(8):659-67; Ingehnan et al. Biochemistry. 1999 Jun. 1;38(22):7040-9.

The amino acid sequence represents a primary structure that encodes the information necessary to form the secondary, tertiary and quaternary structure of the aging associated protein. The software looks at certain parameters encoded by the primary sequence to generate the structural model. These parameters are referred to as “energy terms,” or anisotropic terms and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand and substrate binding regions are identified by the computer system. Three-dimensional structures for potential ligands are generated by entering amino acid or nucleotide sequences or chemical formulas of compounds, as described above. The three-dimensional structure of the potential ligand is then compared to that of the aging associated protein to identify ligands that bind to the aging associated protein, orthologs thereof, etc. Binding affinity between the protein and ligands is determined using energy terms to determine which ligands have an enhanced probability of binding to the protein.

Computer systems are also used to screen for mutations, polymorphic variants, alleles and interspecies homologs and orthologs of the aging associated protein or gene. Such mutations can be associated with disease states. Once the variants are identified, diagnostic assays can be used to identify patients having such mutated genes associated with disease states. Identification of the mutated aging associated protein involves receiving input of a first nucleic acid, e.g., genes disclosed in Tables 1 and 3-7 and orthologs/homologs or conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in aging associated protein, e.g., human genes and mutations associated with disease states. The first and second sequences described above can be saved on a computer readable substrate.

Nucleic acids encoding aging associated proteins can be used with high density oligonucleotide array technology (e.g., GeneChip™) to identify family members and homologs, orthologs, alleles, conservatively modified variants, and polymorphic variants in this invention. In the case where the homologs being identified are linked to a known disease, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

In another aspect, the invention features a computer medium having a plurality of digitally encoded data records. Each data record includes a value representing the level of expression of one or more age-associated gene as described herein in a sample, and a descriptor of the sample. The level of expression can relate to mRNA level and/or protein levels. The record can further include an aging-associated parameter as described herein. The descriptor of the sample can be indicate a subject from which the sample was derived (e.g., a patient, a mutant animal), a treatment (e.g., RNAi treatment), or a location of the sample. In one embodiment, the data record further includes values representing the level of expression of genes and proteins other than an age-associated gene of the invention (e.g., other genes associated with an aging-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining an expression profile of the sample, wherein the profile includes a value representing the level expression of an age-associated gene described herein. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to infer a longevity-associated phenotype in a subject wherein an increase or decrease expression of an age-associated gene described herein is an indication that the subject has or is disposed to having an altered longevity-associated phenotype. The method can be used to monitor a treatment for an aging in a subject. For example, the gene expression profile can be determined for a sample from a subject undergoing treatment. The profile can be compared to a reference profile or to a profile obtained from the subject prior to treatment or prior to onset of the disorder (see, e.g., Golub et al., Science 286:531 (1999)).

In one implementation, qualitative or quantitative information (e.g., expression information or allelic information) about one or more genes, e.g., one or more genes listed in Tables 1, 3, 4, 5, 6, or 7, or homologs or orthologs thereof for a plurality of subjects (e.g., human subjects) is stored in a database. The information can be used evaluate association between the information about the genes and a set of subjects. The subjects can be, e.g., individuals associated with an age-related disorder. The server can compare the information about the genes and evaluate associations (e.g., using statistical tests, e.g., a statistical score that evaluates probability of association with the set of subjects relative to controls or other subjects, or statistical noise).

In another implementation, information about one or more homologs of one or more genes, e.g., one or more genes listed in Tables 1, 3, 4, 5, 6, or 7 for a subject (e.g., a human subject) is stored on a server. A user can send information about the subject (e.g., a patient, a relative of a patient, a sample of gametes (e.g., sperm or oocytes), fetal cells, or a candidate for a treatment) to the server, e.g., from a remote computer that communicates with the server using a network, e.g., the Internet. The server can compare the information about the subject, e.g., to reference information to produce an indication as to the individual propensity for an age-associated disorder. For example, the reference information can be information derived from a reference individual, a particular sequence, or a population of sequences. The indication can be, for example, qualitative or quantitative. An exemplary qualitative indication includes a binary output or a descriptive output (e.g., text or other symbols indicating degree of propensity for an age-associated disorder). An exemplary qualitative indication includes a statistical measure of the probability of developing an age-associated disorder, a score, a percentage, or a parameter for a risk evaluation (e.g., a parameter that can be used in a financial evaluation).

It is also possible for the server to return the indication or information about related subjects (e.g., family members or subjects with a genetic pedigree relationship), e.g., to the user. For example, the server can build a family tree based on a set of related subject. Each individual can be, e.g., assigned a statistical score that evaluates probability of an age-related disorders as a function of one or more genes or factors described herein.

In one method, information about one or more genes described herein (e.g., expression or allelic content)is provided (e.g., communicated, e.g., electronically communicated) to a third party, e.g., a hospital, clinic, a government entity, reimbursing party or insurance company (e.g., a life insurance company). For example, choice of medical procedure, payment for a medical procedure, payment by a reimbursing party, or cost for a service or insurance can be function of the information.

Isolation of Nucleic Acids Encoding Aging Associated Proteins

This invention can include use of routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

Aging associated protein-encoding nucleic acids, polymorphic variants, orthologs, and alleles can be isolated using the C. elegans genes provided herein using, e.g., moderate or low stringent hybridization conditions, by screening libraries, by analyzing a sequence database, and/or by synthetic gene construction. Alternatively, expression libraries can be used to clone aging associated proteins, polymorphic variants, orthologs, and alleles by detecting expressed homologs immunologically with antisera or purified antibodies made against C. elegans or mammalian aging associated proteins or portions thereof or by complementation, e.g., of a C. elegans phenotype. In a preferred embodiment, human nucleic acid libraries are screened for homologs of C. elegans genes or proteins that are associated with aging.

To make a cDNA library, one can choose a source that is rich in the RNA of choice. The mRNA is then made into cDNA using reverse transcriptase, ligated into a recombinant vector, and transfixed into a recombinant host for propagation, screening and cloning. Methods for making and screening cDNA libraries are well known (see, e.g. Gubler & Hoffman, Gene 25:263-269 (1983); Sambrook et al., supra; Ausubel et al., supra).

For a genomic library, the DNA is extracted from the tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro. Recombinant phage are analyzed by plaque hybridization as described in Benton & Davis, Science 196:180-182 (1977). Colony hybridization is carried out as generally described in Grunstein et al., Proc. Natl. Acad. Sci. USA., 72:3961-3965 (1975).

An alternative method of isolating aging associated protein-encoding nucleic acid and their orthologs, alleles, mutants, polymorphic variants, and conservatively modified variants combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of aging associated protein-encoding genes directly from mRNA, from cDNA, from genomic libraries or cDNA libraries. Degenerate oligonucleotides can be designed to amplify homologs and orthologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of aging associated protein encoding mRNA in physiological samples, for nucleic acid sequencing, or for other purposes. Genes amplified by the PCR reaction can be purified from agarose gels and cloned into an appropriate vector.

Gene expression of aging associated proteins can also be analyzed by techniques known in the art, e.g., reverse transcription and amplification of mRNA, isolation of total RNA or poly A⁺ RNA, northern blotting, dot blotting, in situ hybridization, RNase protection, high density polynucleotide array technology, e.g., and the like.

Nucleic acids encoding aging associated proteins can be used with high density oligonucleotide array technology (e.g., GeneChip™) to identify aging associated proteins, orthologs, alleles, conservatively modified variants, and polymorphic variants in this invention. In the case where the homologs and orthologs being identified are linked to modulation of aging associated proteins, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al, Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

The gene for aging associated proteins are typically cloned into intermediate vectors before transformation into prokaryotic or eukaryotic cells for replication and/or expression. These intermediate vectors are typically prokaryote vectors, e.g., plasmids, or shuttle vectors.

Expression in Prokaryotes and Eukaryotes

To obtain high level expression of a cloned gene, such as those cDNAs encoding aging associated proteins, one typically subclones aging associated protein encoding nucleic acids into an expression vector that contains a strong promoter to direct transcription, a transcription/translation terminator, and a ribosome binding site for translational initiation. Suitable bacterial promoters are well known in the art and described, e.g., in Sambrook et al., and Ausubel et al, supra. Bacterial expression systems for expressing aging associated proteins are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of aging associated protein encoding nucleic acid in host cells. A typical expression cassette thus contains a promoter operably linked to the nucleic acid sequence encoding aging associated proteins and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. Generally, high level expression is obtained from inducible promoters only in the presence of the inducing agent; basal expression levels are minimal. Inducible expression vectors are often chosen if expression of the protein of interest is detrimental to eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with mitochondrial respiratory chain protein encoding sequences and glycolysis protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of aging associated proteins, which are then purified using standard techniques (see, e.g., Colley et al., J. Biol. Chem. 264:17619-17622 (1989); Guide to Protein Purificationt, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing aging associated proteins.

After the expression vector is introduced into the cells, the transfected cells are cultured under conditions favoring expression of aging associated proteins, which is recovered from the culture using standard techniques identified below.

Expression vectors with appropriate regulatory sequences can also be used to express a heterologous gene in a nematode. In one example, the expression vector is injected in the gonad of the nematode, and the vector is incorporated, e.g., as an extra-chromosomal array in progeny of the nematode. The vector can further include a second gene (e.g., a marker gene) that indicates the presence of the vector. For example, the heterologous gene can be a mammalian gene, e.g., a mammalian cDNA, or a fragment thereof.

Purification of Aging Associated Proteins

Either naturally occurring or recombinant aging associated proteins can be purified for use in functional assays. Naturally occurring aging associated proteins can be purified, e.g., from human tissue. Recombinant aging associated proteins can be purified from any suitable expression system.

Aging associated proteins may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant aging associated proteins are being purified. For example, proteins having established molecular adhesion properties can be reversible fused to aging associated proteins. With the appropriate ligand, aging associated proteins can be selectively adsorbed to a purification column and then freed from the column in a relatively pure form. The fused protein is then removed by enzymatic activity. Finally, aging associated proteins could be purified using immunoaffinity columns.

A. Purfication of Aging Associated Proteins From Recombinant Bacteria

Recombinant proteins are expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of aging associated protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. Aging associated proteins are separated from other bacterial proteins by standard separation techniques, e.g., with Ni-NTA agarose resin.

Alternatively, it is possible to purify aging associated proteins from bacteria periplasm. After lysis of the bacteria, when the aging associated proteins are exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Standard Protein Separation Techniques for Purifying Aging Associated Proteins

Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

Size Differential Filtration

The molecular weight of the aging associated proteins can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatograhy

The aging associated proteins can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Immunological Detection of Aging Associated Proteins

In addition to the detection of aging associated genes and gene expression using nucleic acid hybridization technology, one can also use immunoassays to detect aging associated proteins of the invention. Such assays are useful for screening for modulators of aging associated proteins, e.g., for regulation of lifespan, as well as for therapeutic and diagnostic applications. Immunoassays can be used to qualitatively or quantitatively analyze aging associated proteins. A general overview of the applicable technology can be found in Harlow & Lane, Antibodies: A Laboratory Manual (1988).

Methods of producing polyclonal and monoclonal antibodies that react specifically with the aging associated proteins are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A number of immunogens comprising portions of aging associated proteins may be used to produce antibodies specifically reactive with an aging associated protein. For example, recombinant protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-specific proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular ortholog, such as a human ortholog, can also be made, by subtracting out other cross-reacting orthologs from a species such as a non-human mammal.

Once the specific antibodies against aging associated proteins are available, the protein can be detected by a variety of immunoassay methods. In addition, the antibody can be used therapeutically as aging associated protein modulators, e.g., to enhance and extend lifespan or to prevent premature aging. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

It is also possible to use protein arrays to detect an aging associated protein, e.g., to concurrently detect a plurality of aging associated proteins. Exemplary methods for producing protein arrays are provided in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 0/98534, WO01/83827, WO02/12893, WO 00/63701, WO 01/40803 and WO 99/51773. In some implementations, polypeptides (including peptides) are spotted onto discrete addresses of the array, e.g., at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

Assays for Modulators of Aging Associated Proteins

A. Assays

Modulation of aging associated proteins and genes can be assessed using a variety of in vitro and in vivo assays, as described herein, and, such assays can be used to test for inhibitors and activators of aging associated proteins. Such modulators of aging associated proteins and genes, which are involved in aging, are useful for enhancing lifespan or treating premature aging. Modulators of aging associated proteins and genes are tested using either recombinant or naturally occurring, preferably C. elegans, mouse, rat, guinea pig, monkey, or human aging associated proteins.

Preferably, the aging associated proteins or genes will have a C. elegans or a mammalian, e.g., a rat, mouse, guinea pig, rabbit, monkey, or human sequence. Alternatively, the aging associated proteins or genes of the assay will be derived from a eukaryote and include an nucleic acid or amino acid subsequence having sequence identity to the C. elegans genes and gene products described herein. Generally, the sequence identity will be at least 30%, 35%, 40%, 45% or 50%, preferably at least 55%, 60%, 65%, 70%, 75%, 80%, 85%, or 90%, most preferably at least 95%.

Measurement of modulation of aging phenotype with aging associated proteins or cells expressing aging associated proteins or genes, either recombinant or naturally occurring, can be performed using a variety of assays, in vitro, in vivo, and ex vivo. A suitable physiological change that affects activity can be used to assess the influence of a test compound on the polypeptide or nucleic acid of this invention. When the functional effects are determined using intact cells or animals, one can also measure a variety of effects such as, increases or decreases in lifespan, cellular proliferation, or in the case of signal transduction, hormone release, transcriptional changes to both known and uncharacterized genetic markers (e.g., northern blots), changes in cell metabolism such as cell growth or pH changes, and changes in intracellular second messengers such as cGMP.

In one embodiment, aging associated protein or gene modulators are assayed in vivo by screening in C. elegans or in a mammalian model system (cellular or animal) for changes in mean and median lifespan.

Some aging associated proteins have measurable enzymatic activity. Thus, enzymatic assays can be performed to identify compounds that modulate the enzymatic activity. Enzymatic activity can encompass a chemical reaction carried out by a protein, as well as binding of substrates, cofactors, regulatory compounds, or ligands to the protein. It may also be useful to monitor the affect of a test compound on other properties of the aging associated protein, e.g., a structural property (e.g., conformation, oligomerization state, stability, mobility, and the like) or a cellular property (e.g., cellular localization, accessibility, clustering, and the like). The protein activity and binding capabilities assayed will depend on the aging associated protein.

The functional activities described herein do not represent all of the enzymatic activities that could be found in aging associated proteins. For example, some aging proteins could act to down regulate transcription of messenger RNA. Still other aging proteins may functional, e.g., as a structural scaffold or adaptor protein, e.g., they may or may not have an enzymatic activity.

Assays to identify compounds with modulating activity can be performed in vitro, e.g., in a test tube, or using isolated membranes, e.g., mitochondrial membranes, or using cellular or mitochondrial extracts. Exemplary assays can include, for example, methods described or referenced in Al-Awqati, Annu. Rev. Cell Biol. 2:179-199 (1986); Brand et al., Biol. Rev. Cambridge Philsophic Soc. 62:141-193 (1987); Capaldi et al., FEBS Lett 138:1-7 (1982); Casey, Biochim. Biophys. Acta 768:319-347 (1984); Erecinska et al., J. Membr. Biol. 70:1-14 (1982); Fillingame, Annu. Rev. Biochem. 49:1079-1113 (1980); Hamamoto, Proc. Natl. Acad. Sci. USA 82:2570-2573 (1985); Hatefi, Annu. Rev. Biochem. 54:1015-1070 (1985); Klingenberg, Trends Biochem. Sci. 4:249-252 (1979); LaNoue et al., Annu. Rev. Biochem. 48:871-922 (1979); Mitchell, Nature 191:144-148 (1961); Prince, Trends Biochem. Sci. 13:159-160 (1988); Slater, Trends Biochem. Sci. 8:239-242 (1983); Srere, Trends Biochem. Sci. 7:375-378 (1982); Tzagoloff, Mitochondria, New York: Plenum (1982); Weiss et al., Biochem. Soc. Thans. 15:100-102 (1987).

For example, the aging associated protein or gene is first contacted with a potential modulator and incubated for a suitable amount of time, e.g., from 0.5 to 48 hours. In one embodiment, aging associated protein or gene expression levels are determined in vitro by measuring the level of protein or mRNA. The level of protein or nucleic acid is measured using immunoassays such as western blotting, ELISA and the like with an antibody that selectively binds to the polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using an aging associated protein promoter operably linked to a reporter gene such as chloramphenicol acetyltransferase, firefly luciferase, bacterial luciferase, β-galactosidase and alkaline phosphatase. Furthermore, the gene or protein of interest can be used as an indirect reporter via attachment to a second reporter such as green fluorescent protein (see, e.g., Mistili & Spector, Nature Biotechnology 15:961-964 (1997)). The reporter construct is typically transfected into a cell. After treatment with a potential modulator, the amount of reporter gene transcription, translation, or activity is measured according to standard techniques known to those of skill in the art.

B. Modulators

The compounds tested as modulators of aging associated proteins and genes can be any small chemical compound, or a biological entity, such as a protein, e.g., an antibody, a sugar, a nucleic acid, e.g., an antisense oligonucleotide, siRNA, dsRNA, RNAi, or a ribozyme, or a lipid. Alternatively, modulators can be genetically altered versions of an aging associated proteins and genes. Typically, test compounds will be small chemical molecules and peptides, or antibodies, antisense molecules, or ribozymes. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihra et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In one embodiment, the invention provides solid phase based in vitro assays in a high throughput format, where the cell or tissue expressing aging associated proteins is attached to a solid phase substrate. In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100- about 1500 different compounds. It is possible to assay many plates per day; assay screens for up to about 6,000, 20,000, 50,000, or 100,000 or more different compounds are possible using the integrated systems of the invention.

Other compounds which have therapeutic or diagnostic use can be designed (e.g., screening may not necessarily be required). Such compounds include, e.g., double-stranded RNAs, ribozymes, antibodies, artificial transcription factors, and so forth. For example, dsRNA can be delivered to cells or to an organism. Endogenous components of the cell or organism can trigger RNA interference (RNAi) which silences expression of genes that include the target sequence. dsRNA can be produced by transcribing a cassette in both directions, for example, by including a T7 promoter on either side of the cassette. Endogenous components of the cell or organism can trigger RNA interference (RNAi) which silences expression of genes that include the target sequence. dsRNA can be produced by transcribing a cassette in both directions, for example, by including a T7 promoter on either side of the cassette. The insert in the cassette is selected so that it includes a sequence complementary to a gene to be attenuated, e.g., a gene listed in Table 1, 3, 4, 5, 6 or 7 or a homolog or ortholog thereof. The sequence need not be full length, for example, an exon, or at least 50 nucleotides, preferably from the 5′ half of the transcript, e.g., within 300 nucleotides of the ATG. See also, the HiScribe™ RNAi Transcription Kit (New England Biolabs, Ma) and Fire, (1999) Trends Genet. 15, 358-363. dsRNA can be digested into smaller fragments. See, e.g., US Patent Application 2002-0086356 and 2003-0084471. In one embodiment, an siRNA is used. siRNAs are small double stranded RNAs (dsRNAs) that optionally include overhangs. For example, the duplex region is about 18 to 25 nucleotides in length, e.g., about 19, 20, 21, 22, 23, or 24 nucleotides in length. Typically the siRNA sequences are exactly complementary to the target mRNA. dsRNAs and siRNAs in particular can be used to silence gene expression in mammalian cells (e.g., human cells). See, e.g., Clemens, et al. (2000) Proc. Natl. Sci. USA 97, 6499-6503; Billy, et al. (2001) Proc. Natl. Sci. USA 98, 14428-14433; Elbashir et al. (2001) Nature 411(6836):494-8; Yang, et al. (2002) Proc. Natl. Acad. Sci. USA 99, 9942-9947.

Artificial transcription factors can be designed to regulate one or more target genes. For example, the factors can be engineered to bind to a regulatory sequence that controls the target gene. A variety of methods can be used to alter the DNA binding specificity of a transcription factor, e.g., by mutating base-contacting residues.

In one embodiment, the artificial trrnscription factor is a chimeric zinc finger protein. The protein can be designed or selected from a library. For example, the protein can be prepare by selection in vitro (e.g., using phage display, U.S. Pat. No. 6,534,261) or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol 19:656; and Wu et al. (1995) Proc. Nat. Acad. Sci. USA 92:344 for, among other things, methods for creating libraries of varied zinc finger domains.

Optionally, the zinc finger protein can be fused to a transcriptional regulatory domain, e.g., an activation domain to activate transcription or a repression domain to repress transcription. The zinc finger protein can itself be encoded by a heterologous nucleic acid that is delivered to a cell or the protein itself can be delivered to a cell (see, e.g., U.S.. The heterologous nucleic acid that includes a sequence encoding the zinc finger protein can be operably linked to an inducible promoter, e.g., to enable fine control of the level of the zinc finger protein in the cell.

In one aspect, the invention provides a method that includes identifying a plurality of genes that are similarly regulated in a cell or organism with altered lifespan regulation (e.g., due to one or more mutations, RNAi, a therapeutic treatment, a disease or disorder, or combinations thereof), identifying one or more DNA sites that can be used to regulate at least two genes of the plurality of genes, and preparing an artificial transcription factor that regulates the at least two genes.

C. Further Exemplary Cellular and Organismal Assays

A test compound identified by a method described herein can also be evaluated for modulation of an age related disorder. Alzheimer's Disease and Huntington's dieases provide two examples of impelementations for studying use of such compounds for age-related disorders

Alzheimer's Disease (AD) is a complex neurodegenerative disease that results in the irreversible loss of neurons. It provides merely one example of a neurodegenerative disease that has symptoms caused at least in part by protein aggregation. Clinical hallmarks of Alzheimer's Disease include progressive impairment in memory, judgment, orientation to physical surroundings, and language. Neuropathological hallmarks of AD include region-specific neuronal loss, amyloid plaques, and neurofibrillary tangles. Amyloid plaques are extracellular plaques containing the 13 amyloid peptide (also known as Aβ, or Aβ42), which is a cleavage product of the β-amyloid precursor protein (also known as APP). Neurofibrillary tangles are insoluble intracellular aggregates composed of filaments of the abnormally hyperphosphorylated microtubule-associated protein, tau. Amyloid plaques and neurofibrillary tangles may contribute to secondary events that lead to neuronal loss by apoptosis (Clark and Karlawish, Ann. Intern. Med. 138(5):400-410 (2003).

A variety of criteria, including genetic, biochemical, physiological, and cognitive criteria, can be used to evaluate AD in a subject. Symptoms and diagnosis of AD are known to medical practitioners. Some exemplary symptoms and markers of AD are presented below.

In one embodiment, it is possible to use a cellular or animal model of AD, e.g., mouse model of AD, e.g., a secondary screen to evaluate a test compound identified by a method described herein. For example, U.S. Pat. No. 6,509,515 describes one such model animal which is naturally able to be used with learning and memory tests. Other animal models are also described in U.S. Pat. Nos. 5,387,742; 5,877,399; 6,358,752; and 6,187,992.

A variety of cell free assays, cell based assays, and organismal assays are available for evaluating polyglutamine aggregation, e.g., Huntingtin polyglutamine aggregation. Some examples are described, e.g., in U.S. 2003-0109476.

Assays (e.g., cell free, cell-based, or organismal) can include a reporter protein that includes a polyglutamine repeat region which has at least 35 polyglutamines. The reporter protein can be easily detectable, e.g., by fluorescence. In one example, PC12 neuronal cell lines that have a construct engineered to express a protein encoded by HD gene exon 1 containing alternating, repeating codons (e.g., repeats of “CAA CAG CAG CAA CAG CAA”) fused to an enhanced GFP (green fluorescent protein) gene can be used. See, e.g., Boado et al. J. Pharmacol. and Experimental Therapeutics 295(1): 239-243 (2000) and Kazantsev et al. Proc. Natl. Acad. Sci. USA 96: 11404-09 (1999). A number of animal model system for Huntington's disease are available. See, e.g., Brouillet, Functional Neurology 15(4): 239-251 (2000); Ona et al. Nature 399: 263-267 (1999), Bates et al. Hum Mol Genet. 6(10):1633-7 (1997); Hansson et al. J of Neurochemistry 78: 694-703; and Rubinsztein, Trends in Genetics, Vol.18, No. 4, pp. 202-209 (a review on various animal and non-human models of HD).

In one embodiment, the animal is a transgenic mouse that can express (in at least one cell) a human Huntingtin protein, a portion thereof, or fusion protein comprising human Huntingtin protein, or a portion thereof, with, for example, at least 36 glutamines (e.g., encoded by CAG repeats (alternatively, any number of the CAG repeats may be CAA) in the CAG repeat segment of exon 1 encoding the polyglutamine tract). These transgenic animals can develop a Huntington's disease-like phenotype. These transgenic mice are characterized by reduced weight gain, reduced lifespan and motor impairment characterized by abnormal gait, resting tremor, hindlimb clasping and hyperactivity from 8 to 10 weeks after birth (for example the R6/2 strain; see Mangiarini et al. Cell 87: 493-506 (1996)). The phenotype worsens progressively toward hypokinesia. The brains of these transgenic mice also demonstrate neurochemical and histological abnormalities, such as changes in neurotransmitter receptors (glutamate, dopaminergic), decreased concentration of N-acetylaspartate (a marker of neuronal integrity) and reduced striatum and brain size. Accordingly, evaluating can include assessing parameters related to neurotransmitter levels, neurotransmitter receptor levels, brain size and striatum size.

D. Solid State and Soluble High Throughput Assays

In one embodiment the invention provides soluble assays using aging associated proteins or genes, or a cell or tissue expressing aging associated proteins or genes, either naturally occurring or recombinant. In another embodiment, the invention provides solid phase based in vitro assays in a high throughput format, where the aging associated protein or gene is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day, assay screens for up to about 6,000, 20,000, 50,000, or more than 100,000 different compounds are possible using the integrated systems of the invention.

The protein of interest, or a cell or membrane comprising the protein of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.). Similarly, any haptenic or antigenic compound can be used in combination with an appropriate antibody to form a tag/tag binder pair.

Synthetic polymers, such as polyurethanes, polyesters, polycarbonates, polyureas, polyarnides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymner arrays is well described in the literature. See, e.g., Merrifield, J. Am. Chem. Soc. 85:2149-2154 (1963) (describing solid phase synthesis of, e.g., peptides); Geysen et al, J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al, Science, 251:767-777 (1991); Sheldon et al., Clinical Chemistry 39(4):718-719 (1993); and Kozal et al, Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

Another example of a high-throughput assay does not require immobilizing a target protein. Such examples include homogenous assays such as fluorescence resonance energy transfer and fluorescence polarization. Spectroscopy can also be used in a variety of ways. Assays can also be used to generate structure-activity relationships (SAR). A method of analyzing an aging associated protein can also include assays that may not be traditionally associated with a particular throughput, e.g., certain NMR binding assays (e.g., SAR by NMR), calorimetry, crystallization, and so forth.

Cellular Transfection and Gene Therapy

The present invention provides the nucleic acids of aging associated proteins for the transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses a protein of the present invention, thereby mitigating the effects of absent, partial inactivation, or abnormal expression of the gene of interest, or increasing lifespan in a subject with normal gene expression. The compositions are administered to a patient in an amount sufficient to elicit a therapeutic response in the patient. An amount adequate to accomplish this is defined as “therapeutically effective dose or amount.”

Such gene therapy procedures have been used to correct acquired and inherited genetic defects. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36(1995); Kremer & Perricaudet, British Medical Bulletin 51(1):31-44 (1995); Haddada et al., in Current Topics in Microbiology and Immunology (Doerfler & Böhm eds., 1995); and Yu et al., Gene Therapy 1:13-26 (1994)).

Pharmaceutical Compositions and Administration

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions owing to diminished or aberrant expression of the protein of choice, the physician evaluates circulating plasma levels of the vector, vector toxicities, progression of the disease, and the production of anti-vector antibodies. In general, the dose equivalent of a naked nucleic acid from a vector is from about 1 fg to 100 μg for a typical 70 kilogram patient, and doses of vectors which include a retroviral particle are calculated to yield an equivalent amount of therapeutic nucleic acid.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

The following examples are provided by way of illustration only and not by way of limitation. Those of skill in the art will readily recognize a variety of noncritical parameters that could be changed or modified to yield essentially similar results.

### Example 1

### Laser Ablation of Germ Cell Precursors to Identify Genes Involved in Aging

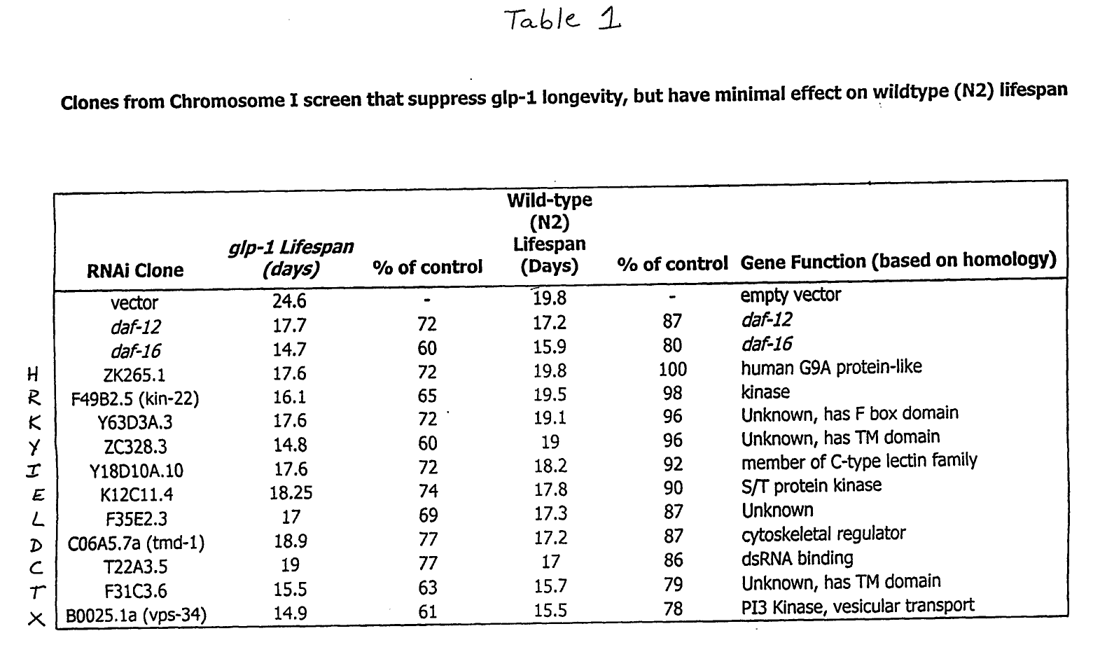

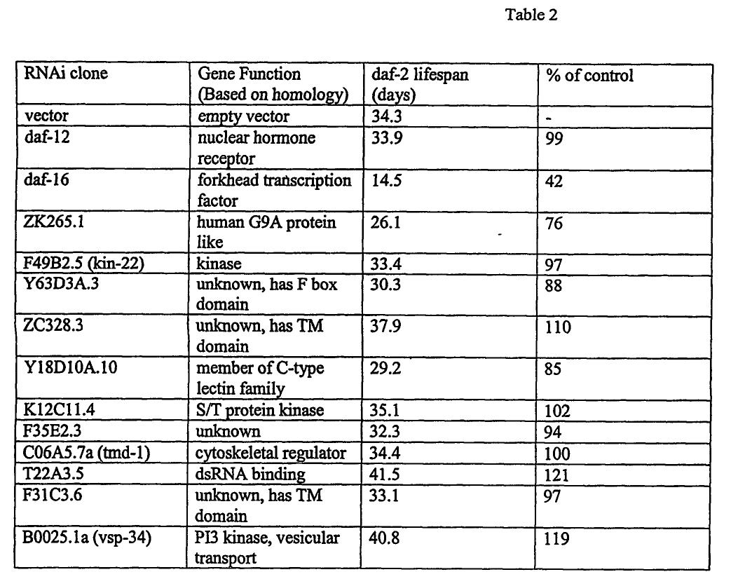

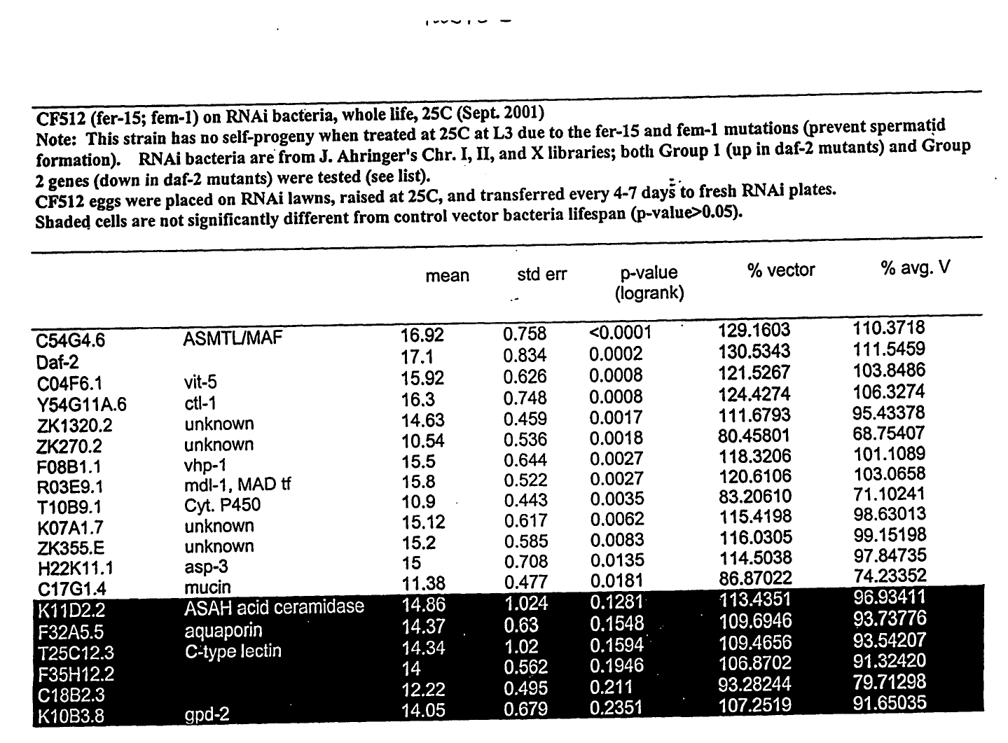

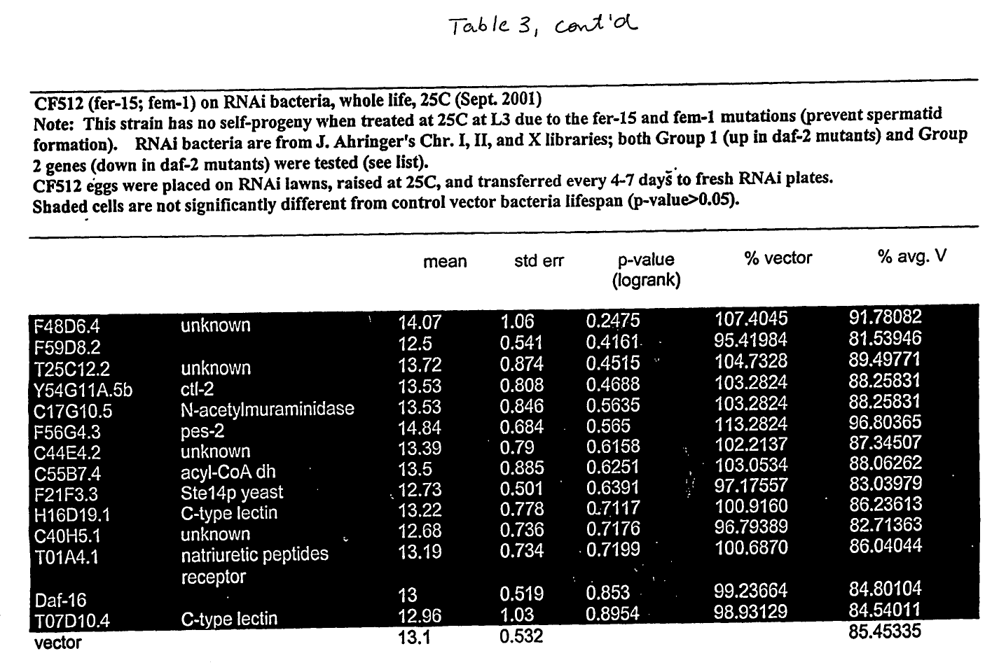

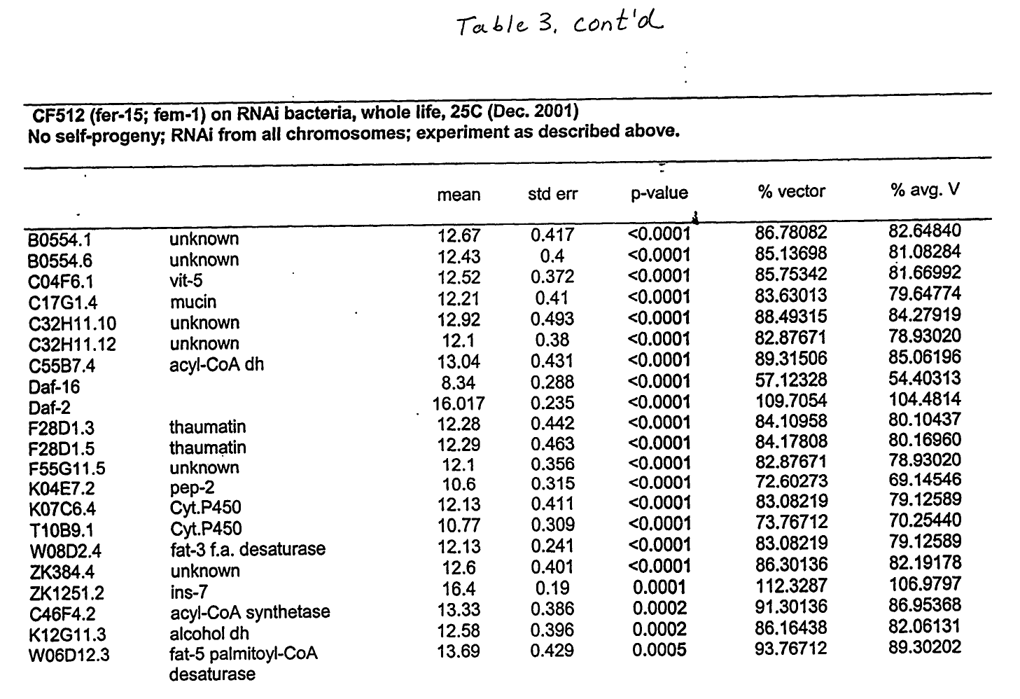

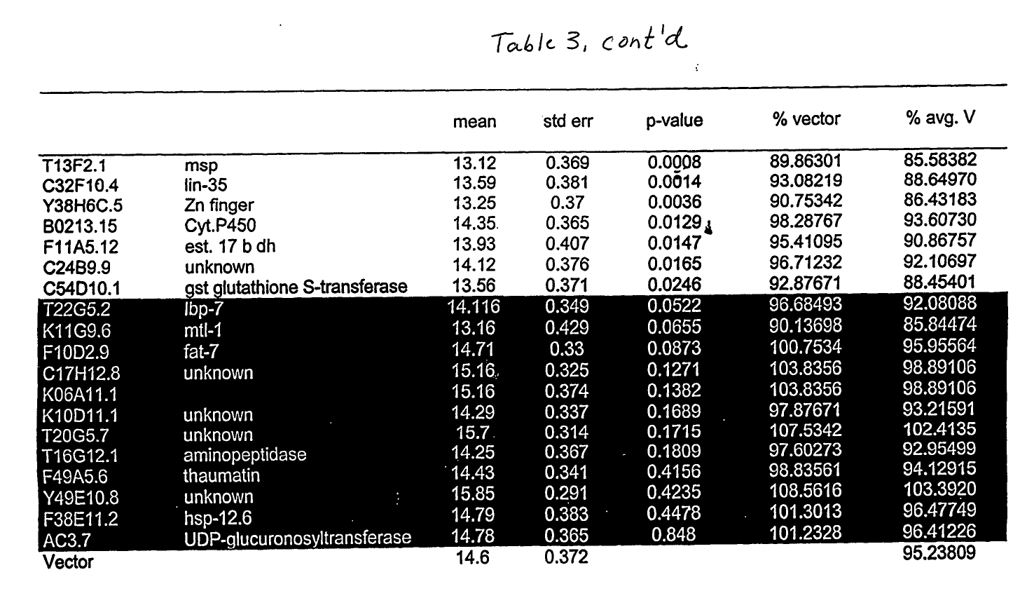

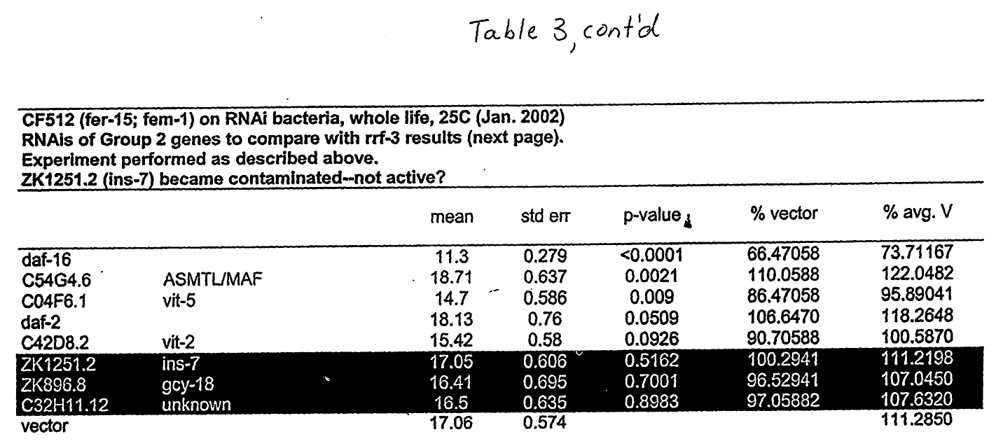

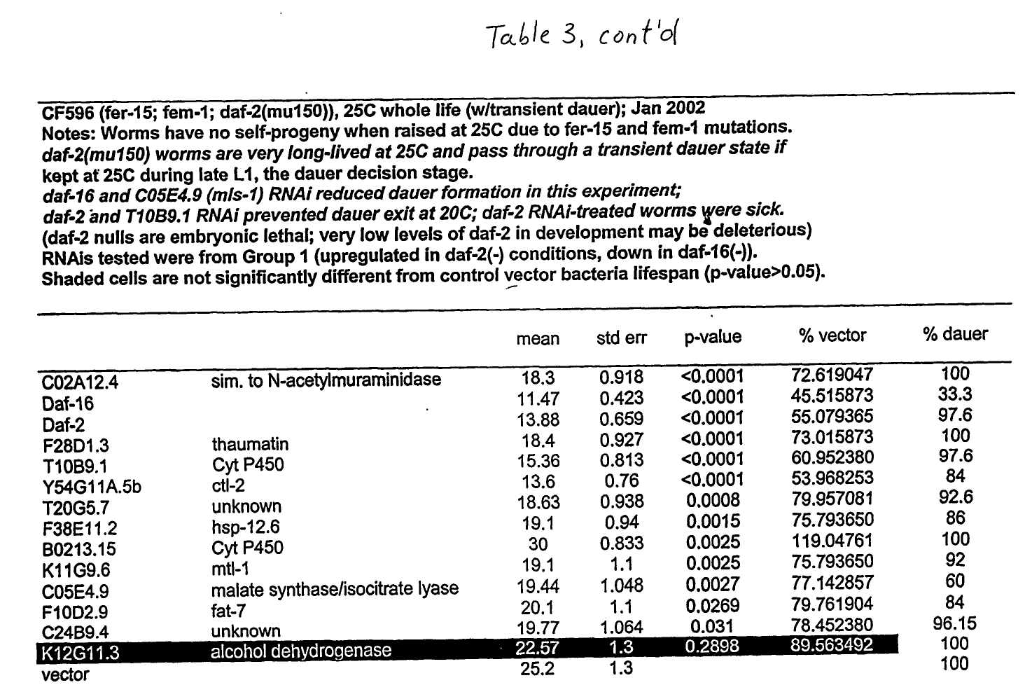

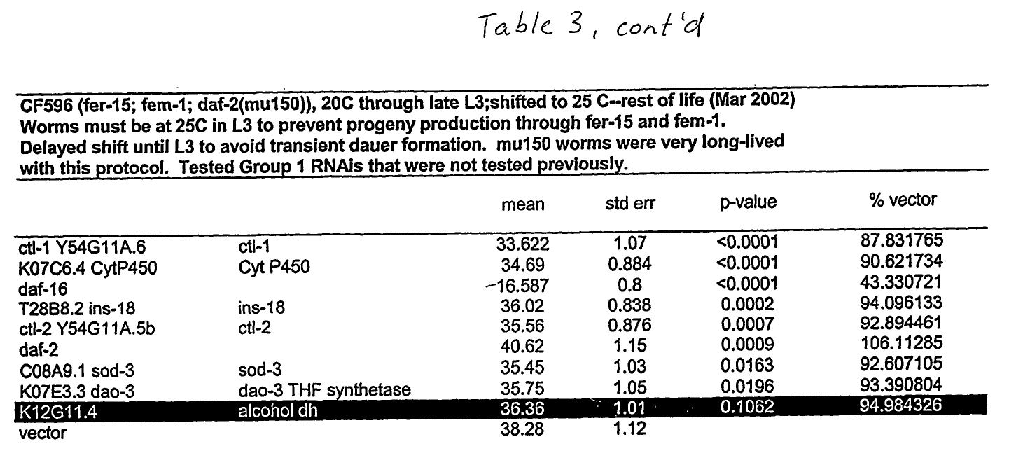

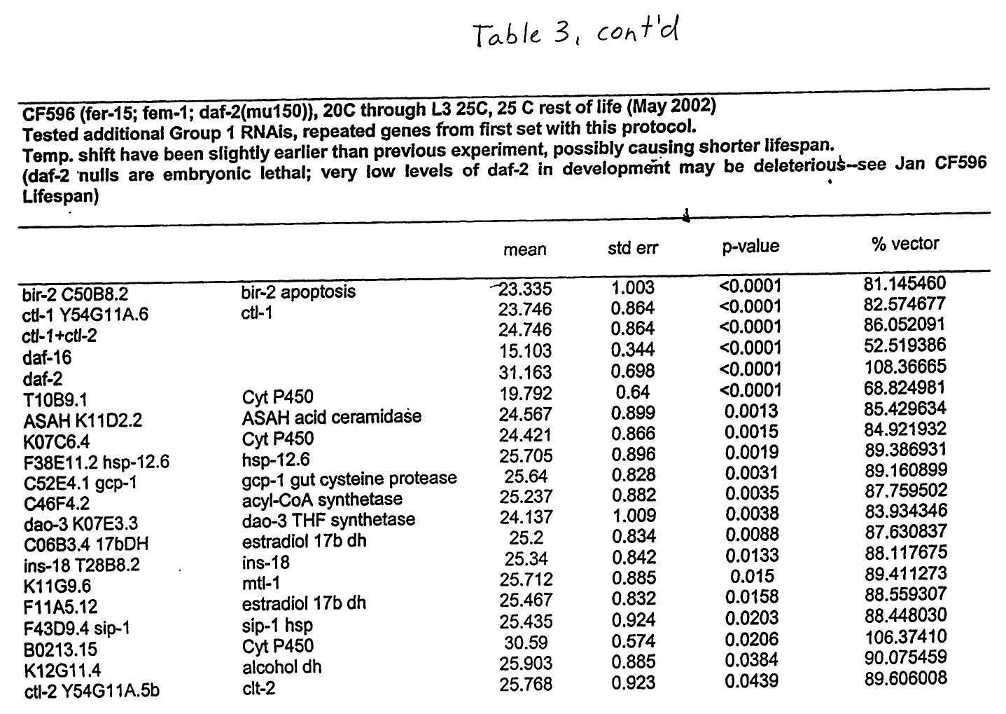

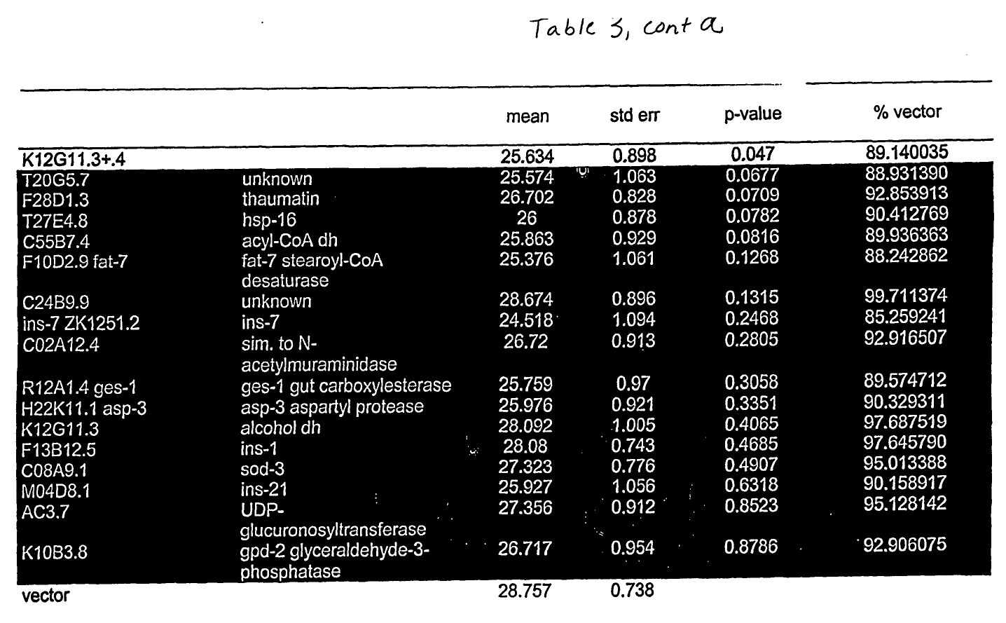

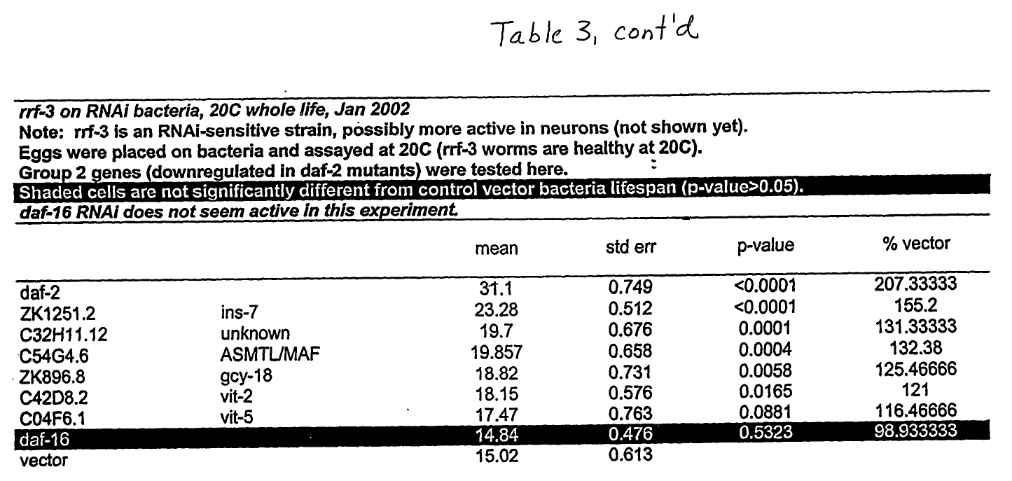

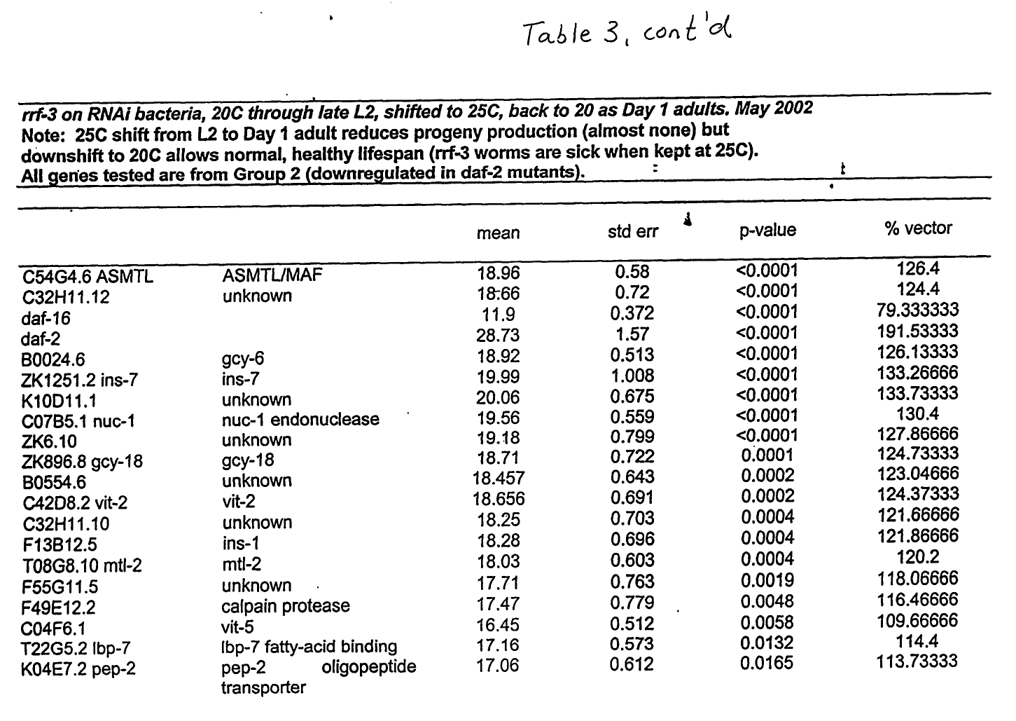

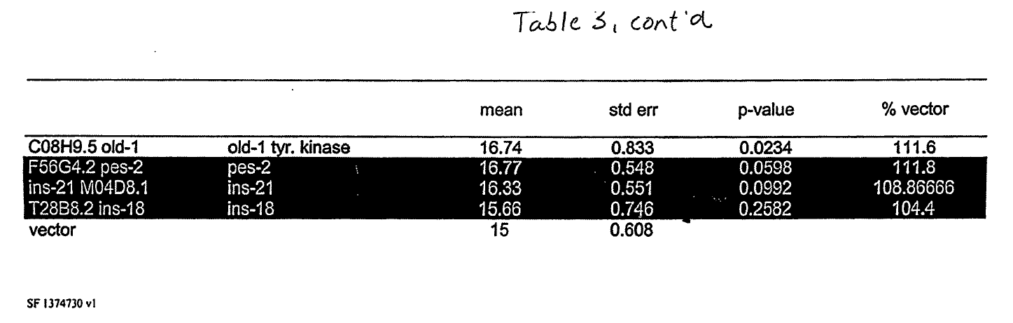

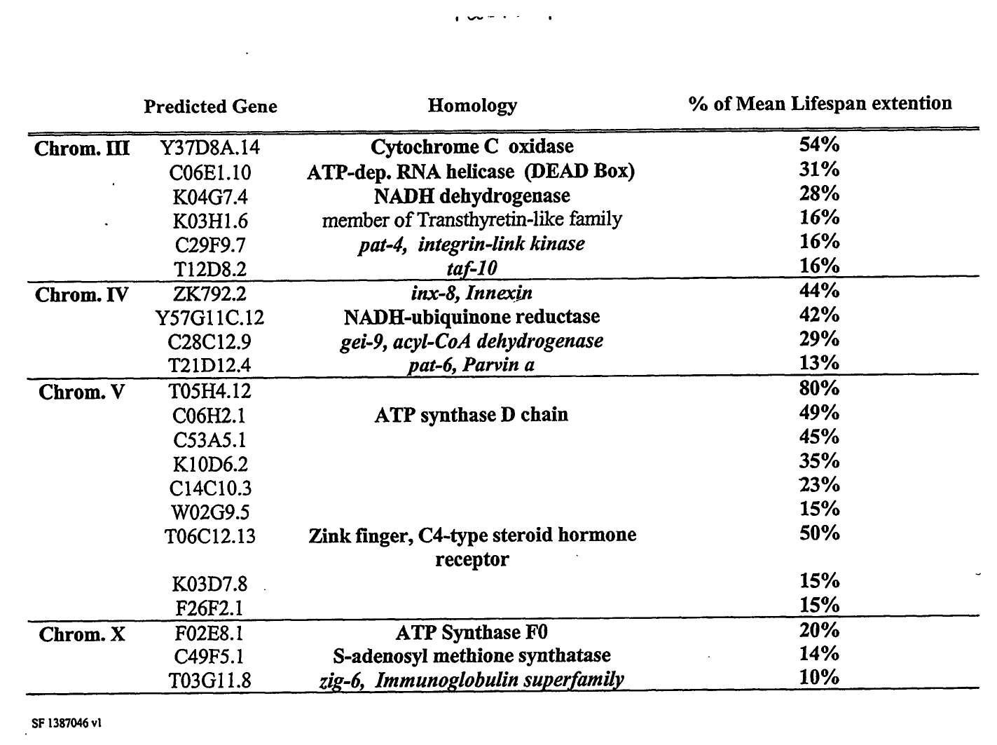

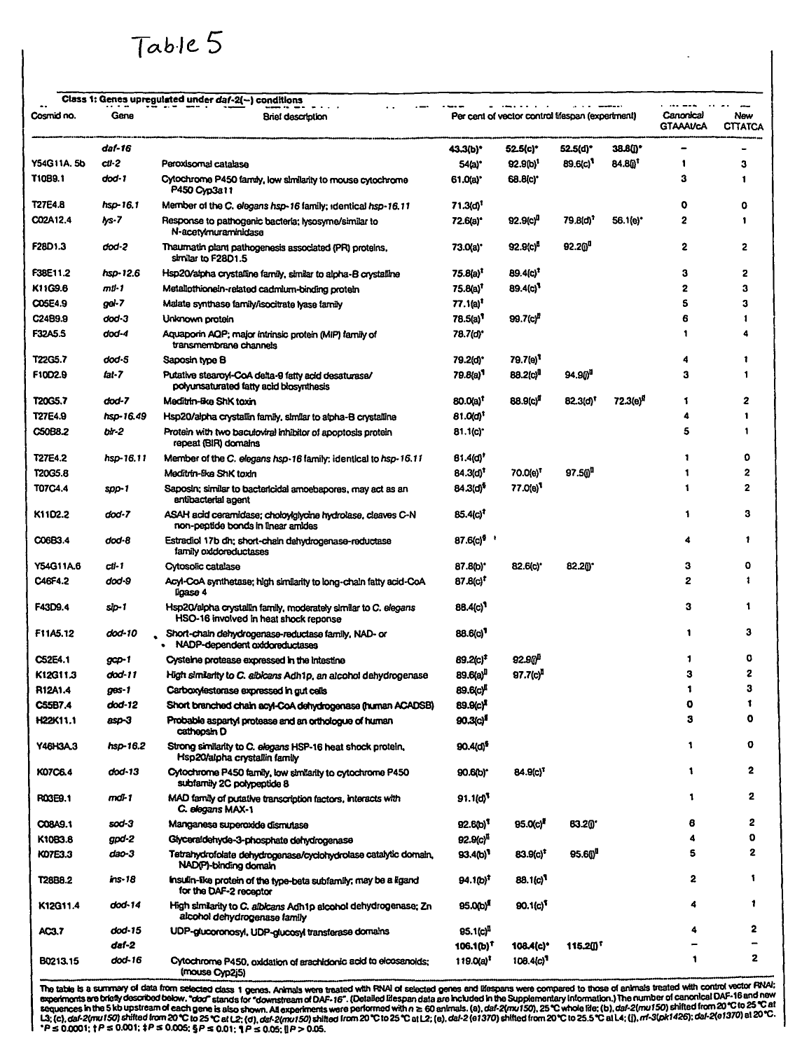

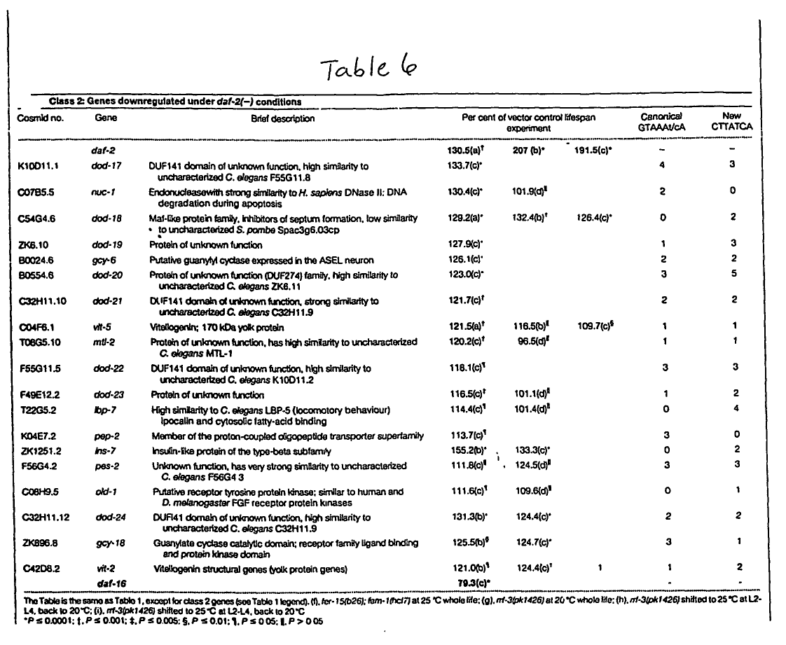

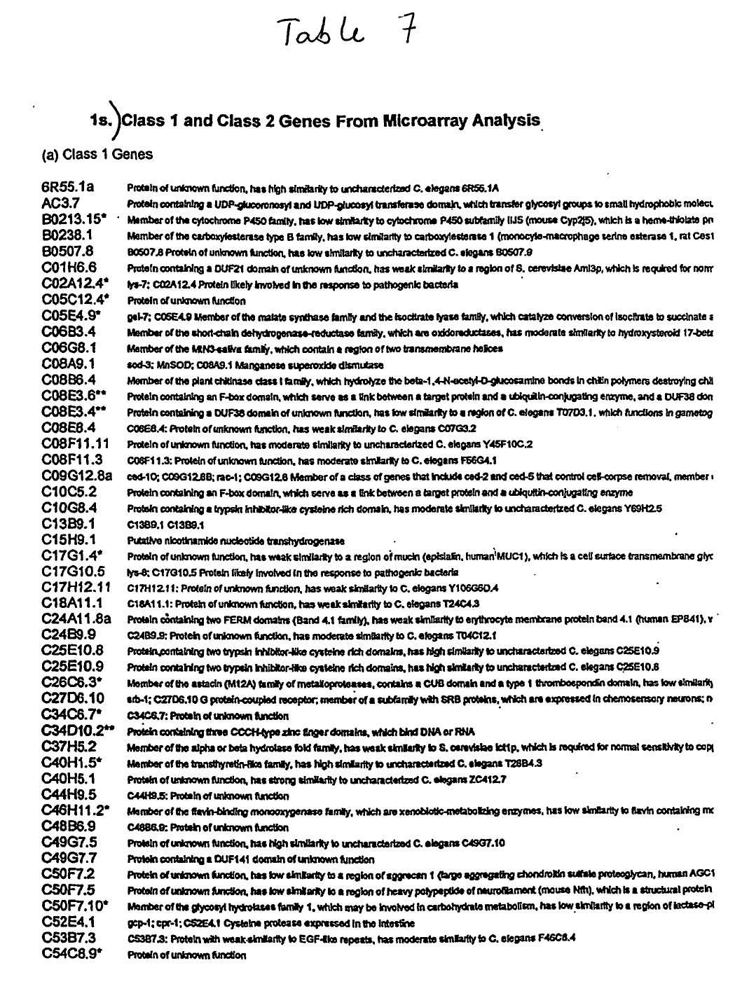

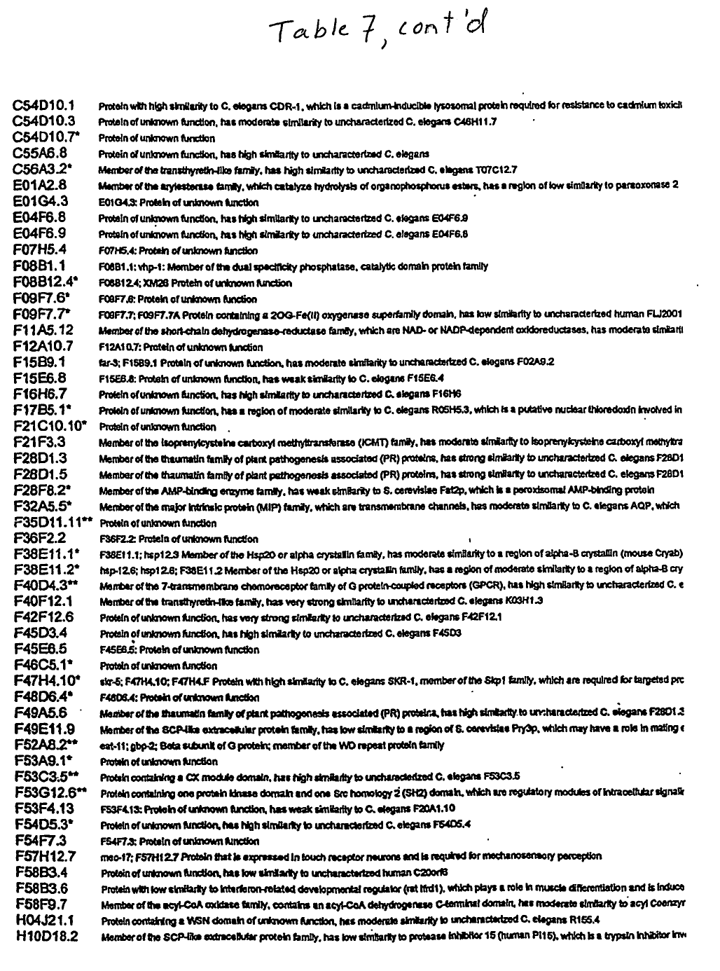

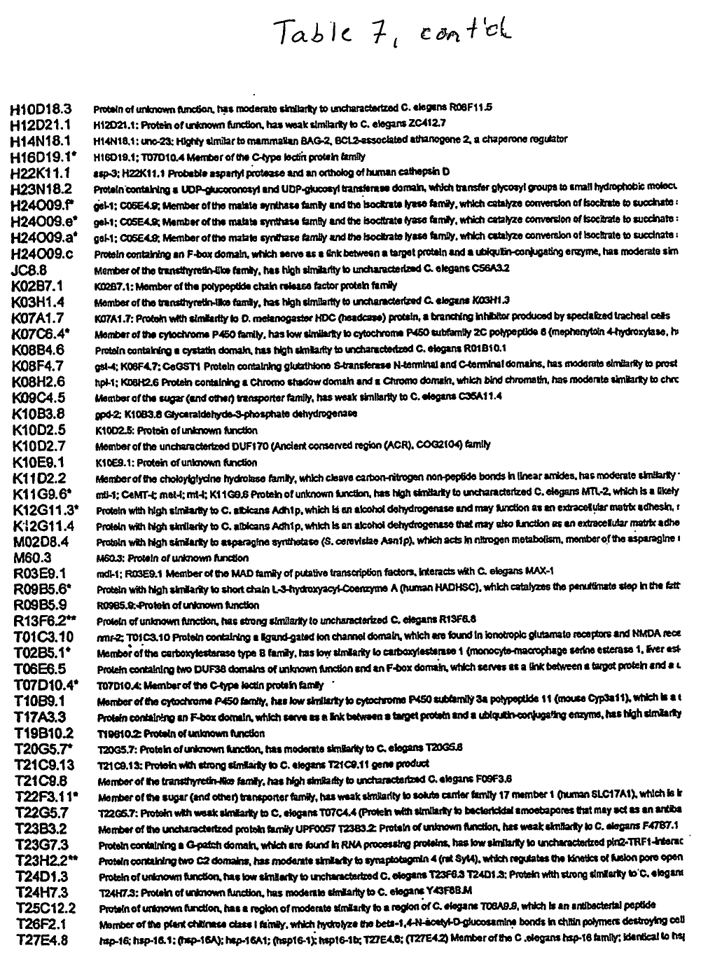

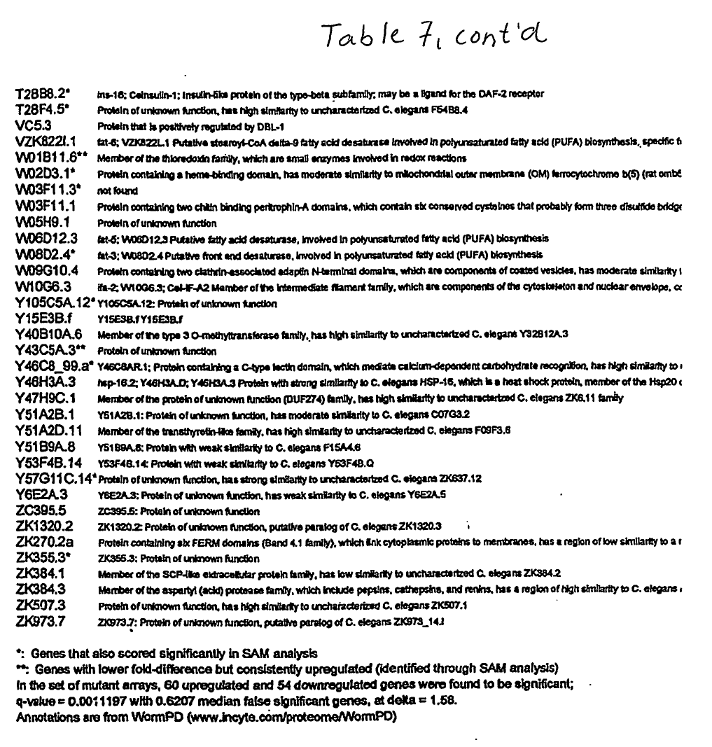

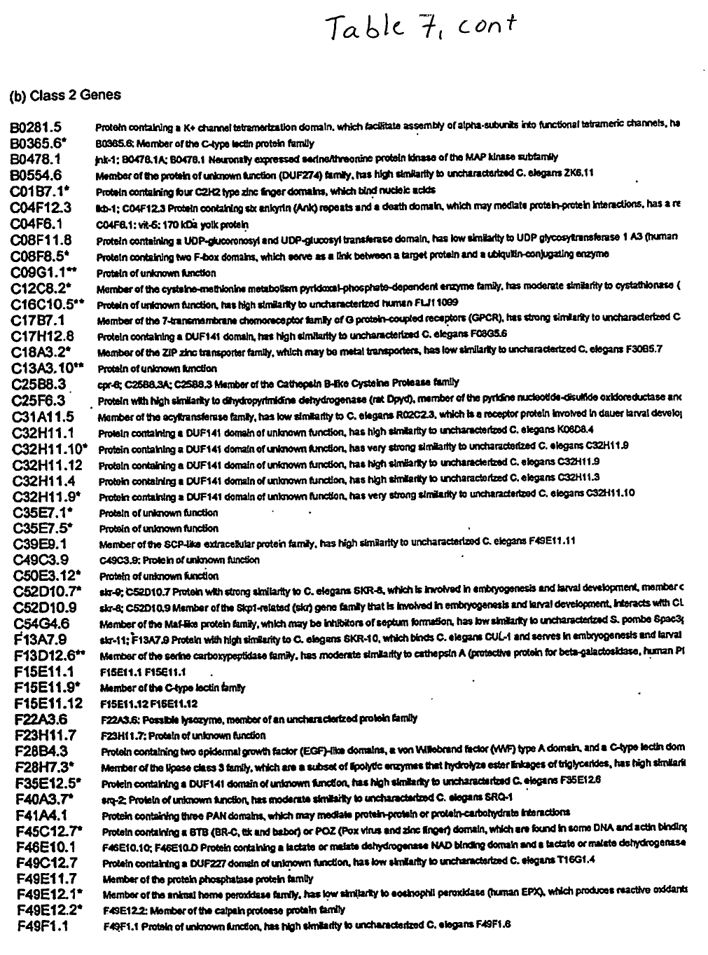

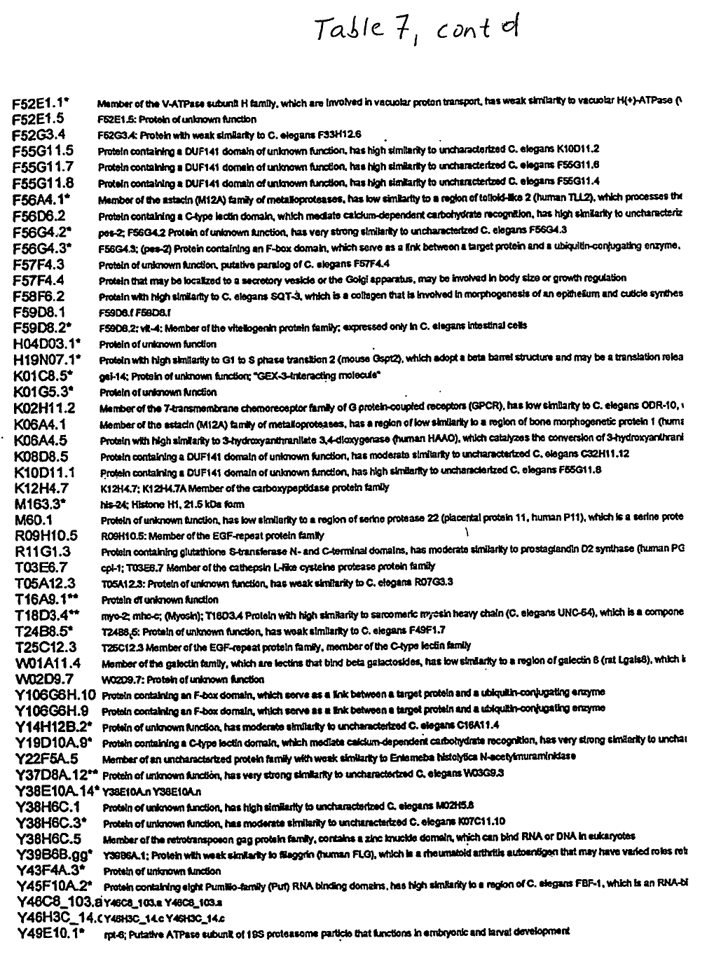

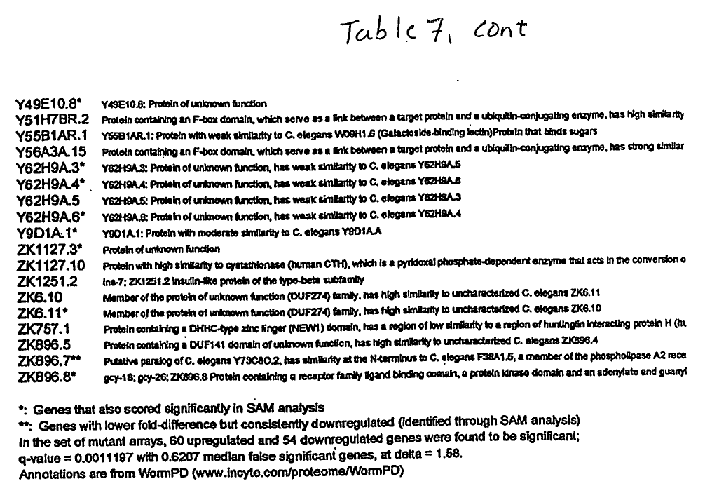

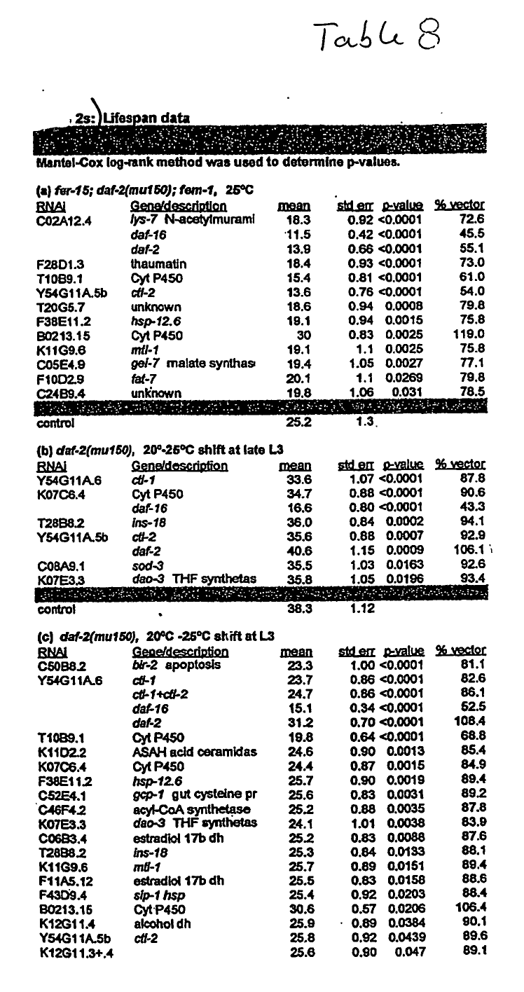

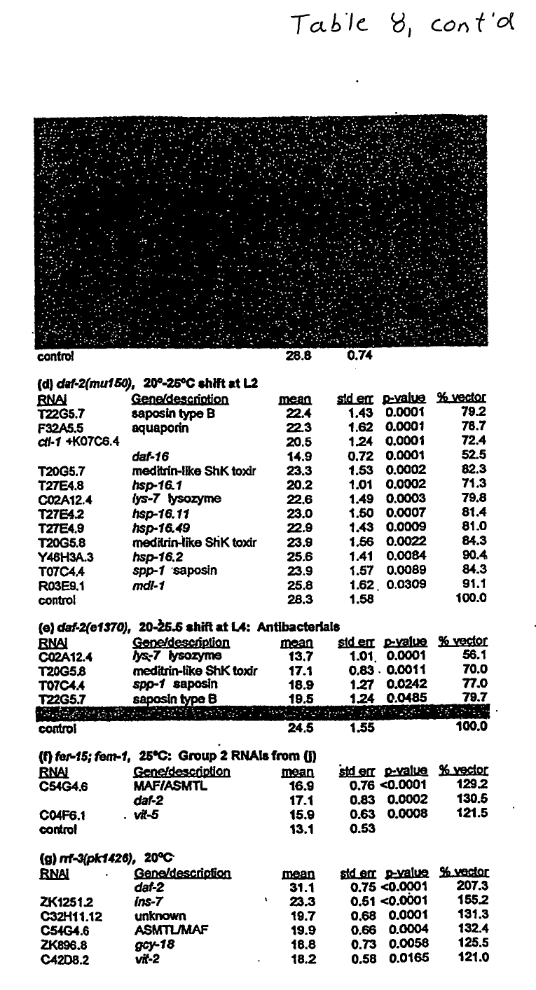

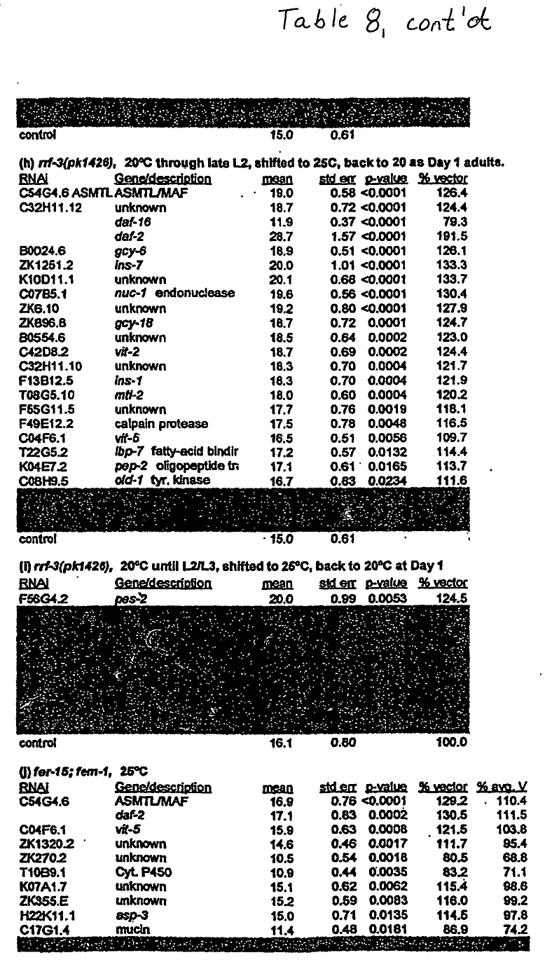

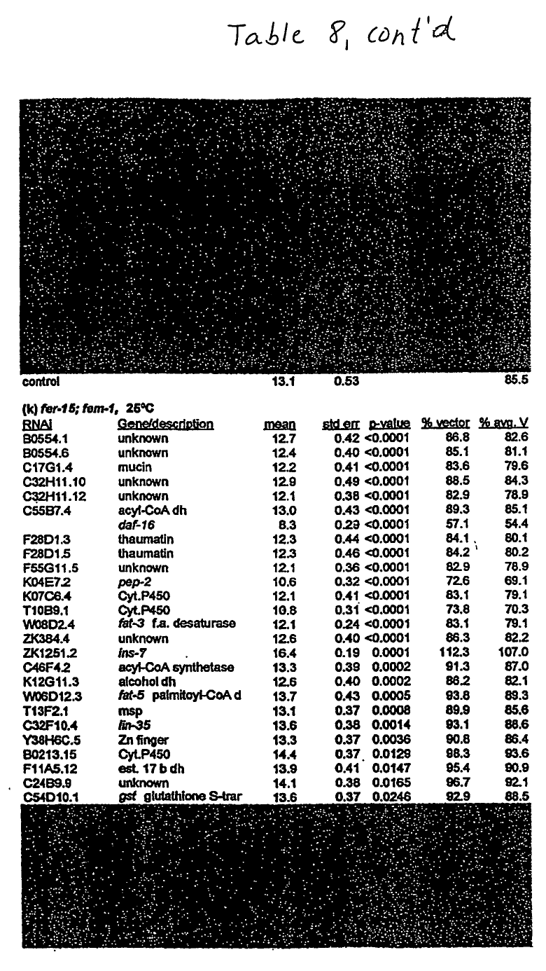

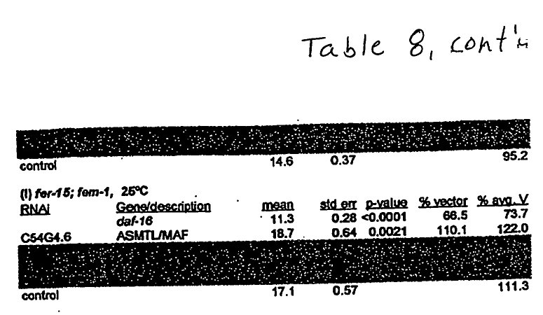

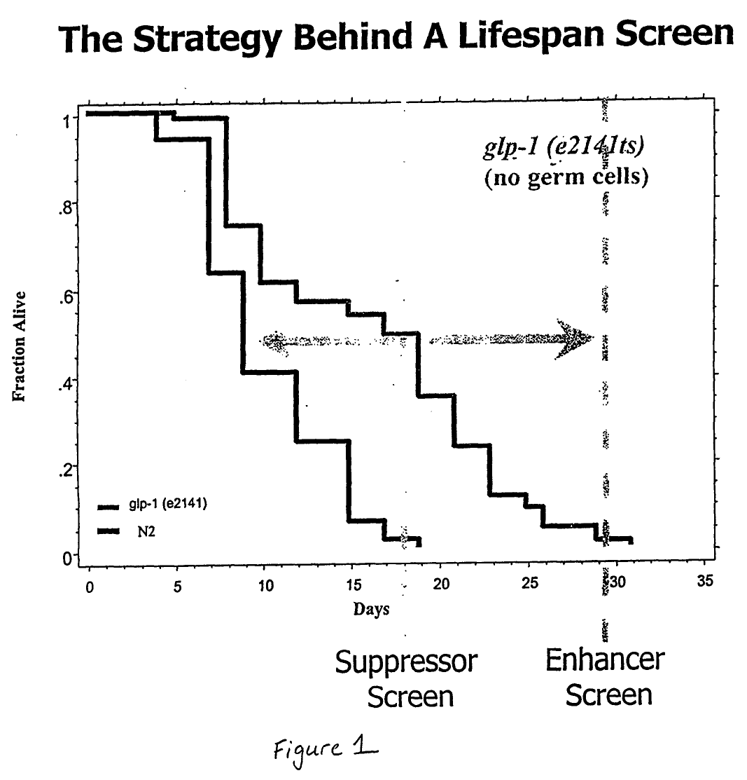

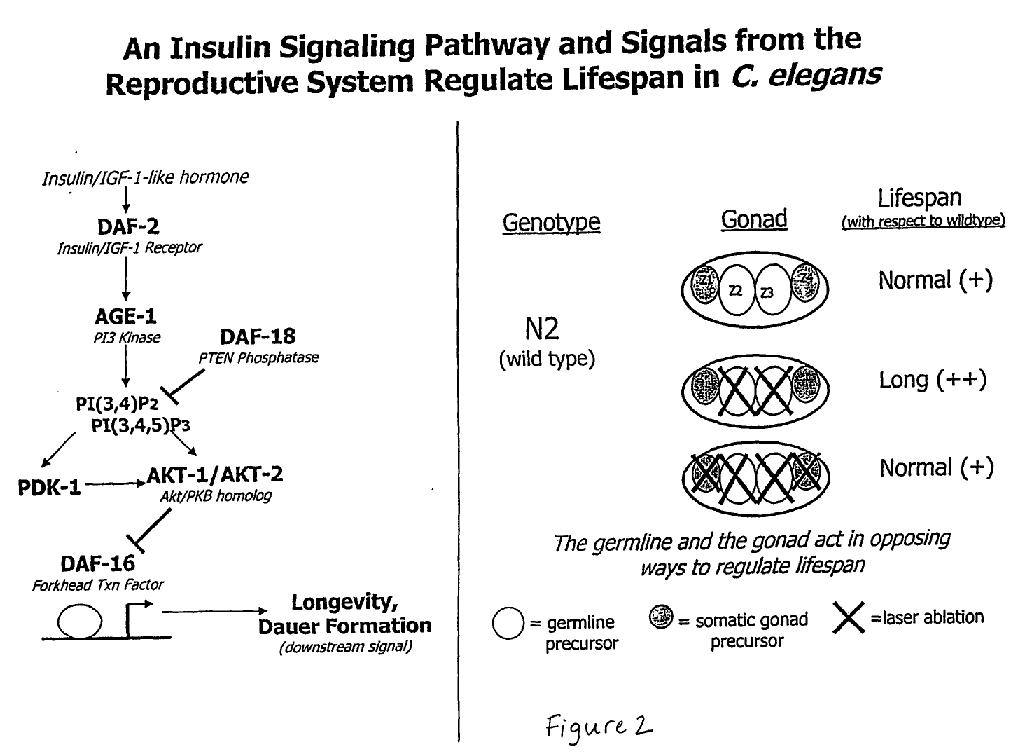

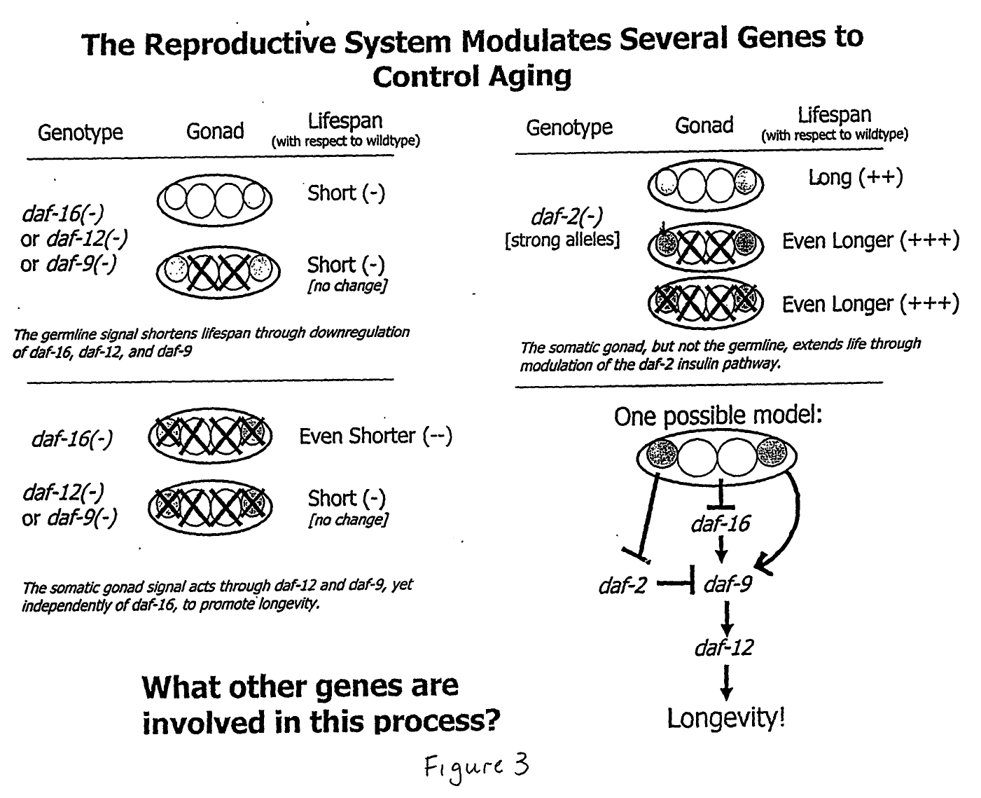

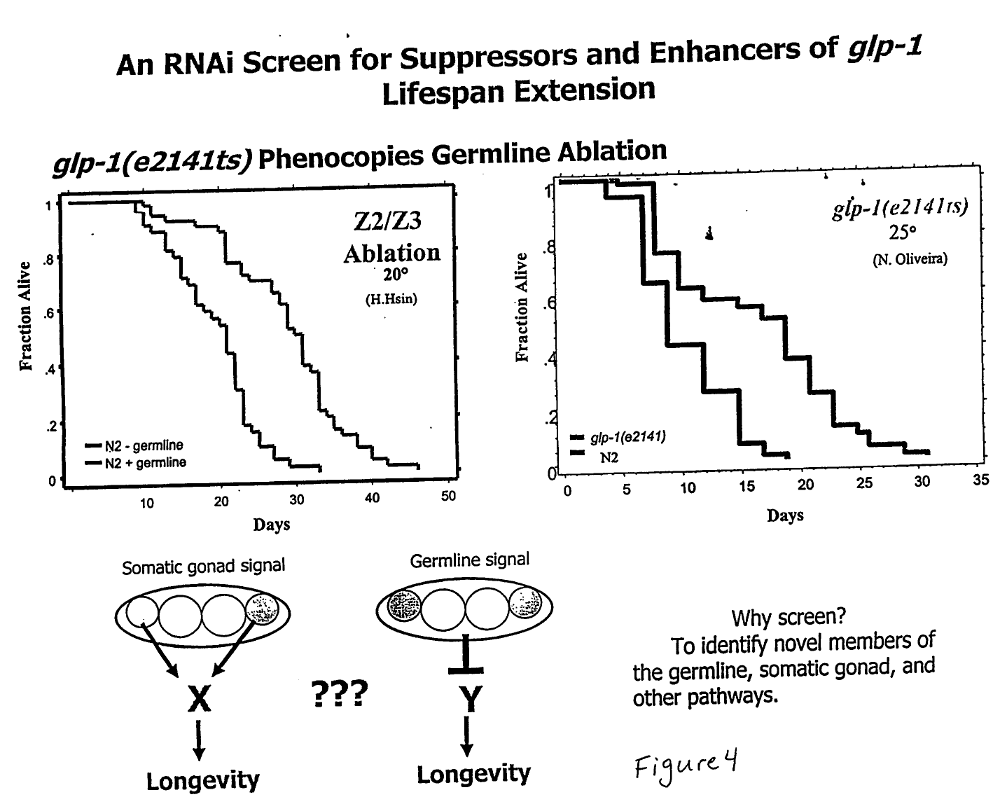

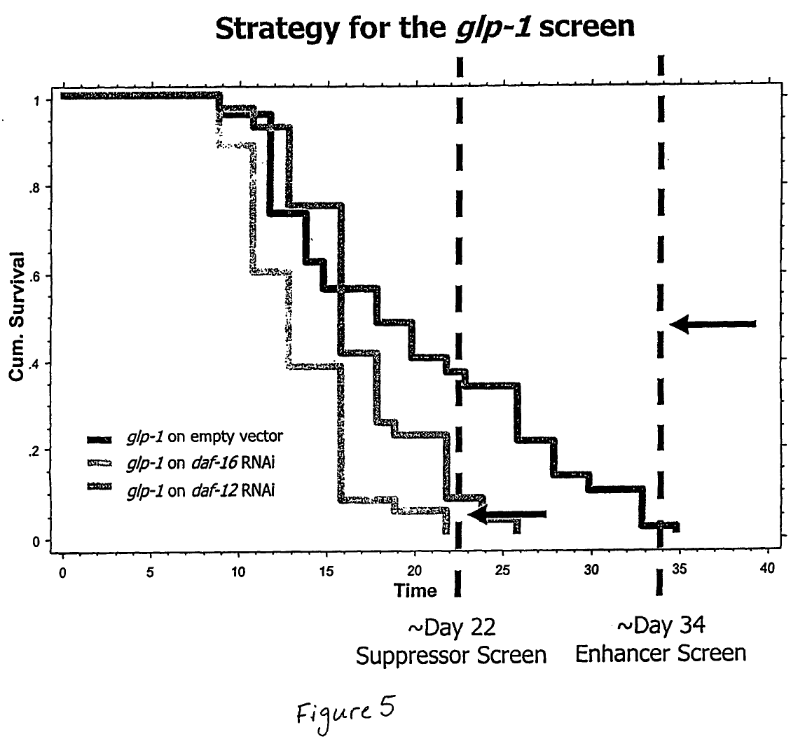

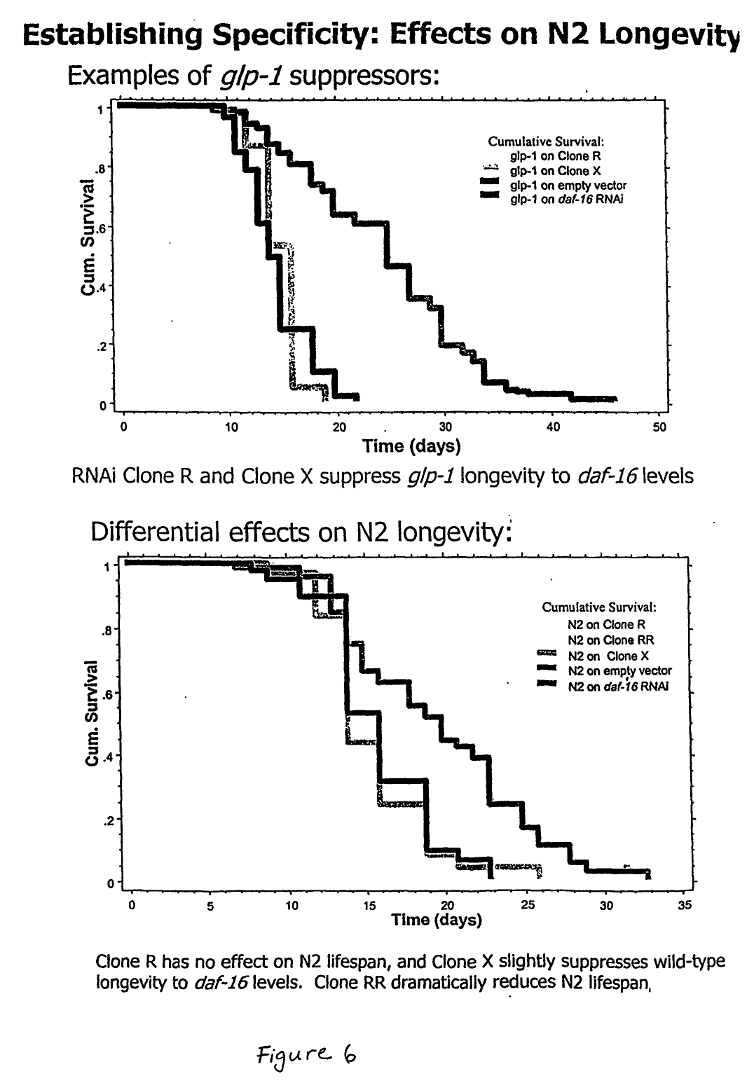

When the germ cell precursors of C. elegans are killed with a laser microbeam, lifespan is extended (see FIGS. 1-6). This extension requires the steroid hormone receptor DAF-12, the cytochrome P450 homolog DAF-9 and the forkhead-family transcription factor DAF-16. The longevity of these animals requires the presence of the somatic gonad. This method can be used as a screen for identifying genes that interact with the reproductive system to regulate the aging of C. elegans.

### Example 2

### Temperature-sensitive Mutation Used to Identify Genes Involved in Aging

The mutant glp-1(e2141) does not produce a germline when grown at high temperature, and as a result, this mutant lives longer than normal (Arantes-Oliveira et al., Science 295:502-505 (2001)). One strategy is to look for bacterial RNAi clones that prevent the lifespan extension of these animals but have only a small effect on wild-type lifespan (see FIGS. 1-6). This strategy can identify genes like daf-16, which are needed in order for germline-ablated animals to live longer than normal. Null mutations in this gene completely suppress the lifespan extension of glp-1 mutants, but have only a small (20%) reduction of wild-type lifespan.

The strategy can also identify genes required for the somatic gonad to extend lifespan. Eleven such genes were identified in a screen of Chromosome 1. The genes are shown in Table 1. Killing the germline precursor cells of normal worms extends lifespan, and this lifespan extension requires the presence of the somatic gonad. If the somatic gonad is killed, the germ cells are unable to survive, but lifespan is not extended. Or, if you kill the somatic gonad and gerinline directly, then lifespan is not affected. These findings suggest that the germline makes a signal that shortens lifespan, and that the somatic gonad makes a counter-acting signal that lengthens lifespan.

The RNAi clones of Table 1 could decrease lifespan either because they prevent germline ablation from lengthening lifespan, or because they prevent the somatic gonad from making its life-extending signal. Previous findings suggest that that the somatic gonad increases lifespan by down-regulating the DAF-2/insulin/IGF-1signaling pathway. If the germline is killed in a long-lived daf-2(e1370) mutant, lifespan is extended. However, if the somatic gonad is also killed, this lifespan extension is not suppressed—the animals still live longer. This is likely because since DAF-2 activity is very low in these mutants, killing the somatic gonad cannot increase DAF-2 activity enough to affect lifespan.

This fact can be used to do a simple test that distinguishes RNAi clones that affect the germline pathway from those that affect the somatic gonad pathway. The ability of RNAi clones shorten the lifespan of daf-2(e1370) mutants is determined (note that these daf-2 animals still have their germ cells, so they are different from animals in which the somatic gonad precursor cells are killed, since this treatment also kills the germline). If the clones don't shorten lifespan, then most likely the reason that they shorten lifespan in daf-2(+) animals is because they up-regulate signaling through the daf-2 pathway. Thus the genes are candidates for functioning in the somatic gonad pathway. Conversely, if the clones shorten the lifespan of daf-2 mutants, then the genes are candidates for those that function in the germline pathway.

This test was carried out on the RNAi clones, and it was found that several did not shorten the lifespan of daf-2(e1370) mutants in a statistically significant way. These are T22A3.5, vps-34, kin-22, Y63D3A.3, ZC328.3, K12C11.4, F35E2.3, tmd-1, and F31C3.6 (see Table 2). These are candidates for genes that function in the somatic gonad pathway to extend lifespan. Two clones did shorten the lifespan of daf-2(e1370) mutants. These are ZK265.1 and Y18D10A.10. These remain candidates for genes that function in the geniline pathway.

### Example 3

### Genes That Act Downstream of DAF-16 to Influence the Lifespan of C. elegans

DAF-16, a FOXO-family transcription factor, influences the rate of ageing of Caenorhabditis elegans in response to insulin and insulin-like growth factor 1 (IGF-1) signaling. Using DNA microarray analysis, we have found that DAF-16 influences expression of a set of genes during early adulthood, the time at which this pathway is known to influence ageing. Here we find that many of these genes influence the ageing process. The insulin/IGF-I pathway functions cell non-autonomously to regulate lifespan, and our findings suggest that it signals other cells, at least in part, by feedback regulation of an insulin/IGF-I homologue. Furthermore, our findings suggest that the insulin/IGF-I pathway ultimately exerts its effect on lifespan by upregulating a wide variety of genes, including cellular stress-response, antimicrobial and metabolic genes, and by downregulating specific life-shortening genes.

The recent discovery that the ageing process is regulated hormonally by an evolutionarily conserved insulin/IGF-I signaling pathway (Tatar et al., Science, 299:1346-1351 (2003); Holzenberger et al., Nature, 421:182-187 (2003); Bluher et al., Science, 299:572-574 (2003)) has provided a powerful entry point for understanding the causes of ageing at the molecular level. The nematode C. elegans lives for only a few weeks; however, animals carrying mutations that decrease insulin and IGF-I signaling, such as daf-2 insuliniIGF-I receptor (Kimura et al., Science, 277:942-946 (1997)) mutations, remain youthfiul and live more than twice as long as normal (Kenyon et al., Nature, 366:461-464 (1993)). The insulin/IGF-I system also regulates reproduction (Kenyon et al., Nature, 366:461-464 (1993); Larsen et al., Genetics, 139:1567-1583 (1995); Gems et al., Genetics, 150:129-155 (1998)) and lipid metabolisrn (Kimura et al., Science, 277:942-946 (1997)), as well as entry into a state of diapause called dauer (Riddle & Albert in C. elegans II (eds Riddle, Blumenthal, Meyer & Priess.) 739-768 (Cold Spring Harbor Laboratory Press, Cold Spring Harbor, N.Y., 1997)). The dauer is an arrested, long-lived juvenile form normally induced by food linitation and also by strong daf-2 mutations (Rddle & Albert, in C. elegans II (eds Riddle, Blumenthal, Meyer & Priess) 739-768 (Cold Spring Harbor Laboratory Press, Cold Spring Harbor, N.Y., 1997)). The DAF-2 pathway regulates reproduction, lipid metabolism, dauer formation and ageing independently of one another (Guarente et al, Nature, 408:255-262 (2000); Gems et al., Curr. Opin. Genet. Dev., 11:287-292 (2001); Dillin et al, Science, 298:830-834 (2002); Wolkow et al, Science, 290:147-150 (2000)). For example, whereas it acts during development to regulate dauer formation, it acts exclusively in the adult to influence ageing (Dillin et al, Science, 298:830-834 (2002)).

The DAF-2 pathway exerts its effects on the animal by influencing downstream gene expression, as the ability of daf-2 mutations (daf-2(−) phenotypes depends on the activity of DAF-16 (Kenyon et al., Nature, 366:461-464 (1993); Larsen et al., Genetics, 139:1567-1583 (1995); Gems et al., Genetics, 150:129-155 (1998); Dillin et al, Science, 298:830-834 (2002); Tissenbaum et al., Genetics, 148:703-717 (1998)), a FOXO family transcription factor (Lin et al., Science, 278:1319-1322 (1997); Ogg et al, Nature, 389:994-999 (1997)). In wild-type animals, the activity of DAF-16 is inhibited by a conserved phosphatidylinositol-3-OH kinase (PI(3)K)/protein kinase D (PDK)/Akt pathway in response to DAF-2 activity (Tatar et al., Science, 299:1346-1351 (2003)).

It should be possible to learn how insulin/IGF-I signaling influences ageing by identifying and characterizing the genes regulated by DAF-16. Some of these genes are predicted to encode or regulate downstream signals or hormones, because daf-2 (and therefore presumably daf-16) functions cell non-autonomously (Wolkow et al, Science, 290:147-150 (2000); Apfeld et al., Cell, 95:199-210 (1998)): removing daf-2 activity from subsets of cells can cause the entire animal to enter the dauer state, or to become a long-lived adult (Apfeld et al., Cell, 95:199-210 (1998)). In addition, DAF-16 is predicted to influence expression of genes whose activities influence the ageing process directly. Animals with reduced DAF-2 pathway activity are resistant to heat and oxidative stress (Tatar et al., Science, 299:1346-1351 (2003); Gems et al., Genetics, 150:129-155 (1998); Guarente et al., Nature, 408:255-262 (2000); Larsen, P. L., Proc. Natl Acad. Sci. USA, 90:8905-8909 (1993); Lithgow et al., J. Gerontol., 49:B270-B276 (1994)) which has suggested that an increased ability to prevent or repair oxidative damage increases lifespan. Consistent with this idea, overexpressing superoxide dismutase can extend the lifespan of Drosophila (Orr et al., Science, 263:1128-1130 (1994); Sun et al., Genetics, 161:661-672 (2002)) and yeast (Longo et al., Arch. Biochem. Biophys., 365:131-142 (1999)). However, this hypothesis has never been tested directly; for example, by asking whether stress response genes are required for the longevity of daf-2 mutants. (An influential report was retracted recently (Taub et al., Nature, 421:764 (2003)).) In this study, we have identified genes that are regulated by DAF-16 and investigated their roles in the ageing process. To do this, we used microarray analysis to identify downstream genes, and then carried out a functional analysis of these genes using RNA interference (RNAi).

Results

DNA Microarray Analysis

We identified genes whose expression changed in insulin/IGF-I pathway mutants using DNA microarrays containing approximately 93% of the predicted C. elegans open reading frames. We did this in two ways. First, we compared the transcriptional profiles of multiple alleles of long-lived daf-2 and age-1/PI(3)K mutants to profiles of wild-type animals and daf-16; daf-2 double mutants on the first day of adulthood. We grouped genes with similar expression patterns by hierarchical clustering of those with at least fourfold expression changes (Eisen et al., Proc. Natl Acad. Sci. USA, 95:14863-14868 (1998)). This allowed us to identify genes that were upregulated or downregulated across the set of arrays. We also identified genes that were regulated in a highly consistent fashion, regardless of the degree to which their expression was changed (Tusher et al., Proc. Nat'l Acad Sci. USA 98:5116-5121 (2001) (Table 7).

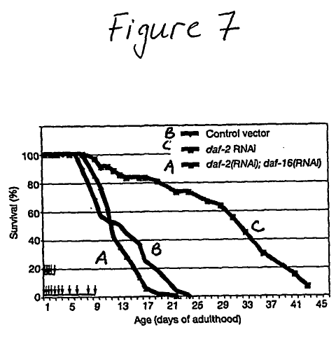

In addition, we reduced daf-2 and daf-16 activity using RNAi, which phenocopies daf-2 and daf-16 mutants11 (FIG. 7). This allowed us to analyze the transcriptional profiles of isogenic and developmentally synchronous animals. We grew a sterile strain (fer-15(b26);fem-1(hc17)) on bacteria expressing daf-2 doublestranded (ds)RNA, both daf-2 and daf-16 dsRNA, or control bacteria, and collected the animals at intervals throughout adulthood (FIG. 7). Because reducing the level of insulin/IGF-I signaling during early adulthood is sufficient to increase lifespan, we carried out an early adult time course (ten time points from 0-48 h of adulthood; FIG. 7) to identify changes that occurred during this period. We also carried out a longer time course (ten time points from 0-192 h of adulthood) to identify changes that occurred as these animals began to age, but before a significant fraction died (FIG. 7).

The Early Ageing Transcriptome is Largely Unaffected

Because mutations in the insulin/IGF-I pathway slow the rate of ageing (Kenyon et al., Nature, 366:461-464 (1993); Garigan et al., Genetics, 161:1101-1112 (2002); Herndon et al., Probl. Cell Differ., 29:113-129 (2000)), we wondered whether reducing daf-2 activity would slow the rate of all age-associated changes in gene expression. To investigate this, we compared the whole-transcriptome profiles of RNAi-treated animals at different ages. In the early adult time course, before the different strains began to differ morphologically (Garigan et al., Genetics, 161:1101-1112 (2002)) (048 h), we found that a subset of genes was expressed differently between animals exposed to daf-2 RNAi and animals exposed to both daf-16 and daf-2 RNAi-we refer to these animals, which do not have long lifespans (FIG. 7), as daf-2(RNAi); daf-16(RNAi) animals. These differences in expression persisted during the longer time course (0-192 h). During this period, the expression of many other genes changed as well; however, most of these age-dependent changes were not different between the daf-2(RNAi) and daf-2(RNAi); daf-16(RNAi) animals. This was surprising, as the tissue morphology of these animals differs significantly by the end of this period (Garigan et al., Genetics, 161:1101-1112 (2002)). Together these findings raised the possibility that the insulin/IGF-I pathway might influence ageing through a relatively small set of physiologically important targets that were differentially expressed even in young adults.

Two Classes of Downstream Genes

We combined the data from our 60 microarrays into a single set and performed hierarchical clustering (Eisen et al., Proc. Natl Acad. Sci. USA, 95:14863-14868 (1998)) (see also Murphy et al., Nature 424:277-283 (2003), herein incorporated by reference in its entirety). We then focused on clusters that showed opposite expression profiles under daf-2 (−) and daf-16 (−) conditions. By examining a variety of mutants in multiple experiments and by performing two longitudinal studies, we were able to eliminate false positives caused by differences in developmental rates and by systematic errors. This approach revealed a relatively small number of differentially expressed daf-2/daf-16-dependent targets.

Two clusters were of particular interest. The firrt contained genes that were induced in DAF-2 pathway mutants and in daf-2(RNAi) animals but repressed in daf-2(RNAi); daf-16(RNAi) animals (class 1). These were candidates for genes that extend lifespan (see also Table 7). Class I genes are upregulated with daf-2 RNAi treatment and in daf-2 pathway mutants, and downregulated with daf-16 RNAi treatment. The second cluster contained genes that displayed the opposite profile (class 2, see also Table 7), and are candidates for genes that shorten lifespan. Class 2 genes are upregulated with daf-16 RNAi treatment and downregulated with daf-2 RNAi treatment and in daf-2 pathway mutants. This approach identified genes previously thought to be regulated by DAF-16, such as the metallothionein homologue mtl-1 (Barsyte et al., FASEB J., 15:627-634 (2001)) and the mitochondrial superoxide dismutase gene sod-3 (Honda et al., FASEB J., 13:1385-1393 (1999)). We carried out polymerase chain reaction with reverse transcription (RT-PCR) of several RNA samples with sod-3- and mtl-1-specific primers, and found that expression of both was increased in daf-2(RNAi) animals, confifining our microarray results.

A Positive Feedback Loop Amplifies Insulin/IGF-I-pathway Activity

In humans, reduced insulin receptor activity in the pancreas reduces insulin production. We found that gene expression of ins-7, which encodes an insulin/IGF-1-like peptide, was repressed in animals with reduced daf-2 activity and elevated in animals with reduced daf-16 activity. ins-7 gene expression was repressed in animals with reduced daf-2 activity, and elevated in animals with reduced daf-16 activity. More than 35 insulin-like genes have been identified in the C. elegans genome (Eisen et al., Proc. Natl Acad. Sci. USA, 95:14863-14868 (1998); Gregoire et al., Biochem. Biophys. Res. Commun., 249:385-390 (1998); Pierce et al., Genes Dev., 15:672-686 (2001); Kawano et al., Biochem. Biophys. Res. Commun., 273:431-436 (2000)) and 23 of these insulin-like genes were represented on our microarrays. A number of insulin-like peptides have been implicated in DAF-2 regulation (Pierce et al., Genes Dev., 15:672-686 (2001); Kawano et al., Biochem. Biophys. Res. Commun., 273:431-436 (2000); Li et al., Genes Dev., 17:844-858 (2003)). To investigate whether ins-7 might function as a DAF-2 agonist, we inhibited its activity using RNAi. We found that ins-7 RNAi increased the lifespan of daf-2 (+)rrf-3(pkl426) animals significantly (FIG. 8a), but was unable to further extend the lifespan of long-lived daf-2 (mu150) animals (see Table 8). Furthermore, ins-7 RNAi increased the frequency of dauer formation in daf-2(e1370ts) animals (FIG. 8b). ins-7 also extends the lifespan offer-15; fem- animals (Table 8). Thus INS-7 behaved as expected for a DAF-2 agonist.

The regulatory properties of ins-7 suggest that it might contribute to the non-autonomy of daf-2 function. INS-7 behaves as a DAF-2 agonist and is part of a positive feedback loop which amplifies DAF-2 pathway activation. In this model, if daf-2 gene activity is removed from cells that normally express ins-7, the level of ins-7 expression will fall, which in turn will lower the level of INS-7 available to activate the DAF-2 receptor present on wild-type cells (FIG. 8c). In our model, when DAF-2 is active, DAF-16 activity is inhibited and ins-7 is expressed, allowing further DAF-2 activation. When DAF-2 activity is reduced, DAF-16 is activated and ins-7 expression is inhibited. In addition, the regulatory properties of INS-7 might contribute to an interesting phenomenon that occurs in nature. When a population of wild-type juvenile animals is confronted with a diminishing food supply (or when temperature-sensitive daf-2 mutants are grown at a semi-permissive temperature), some but not all individuals enter the dauer state. It is interesting that under these threshold conditions, one does not observe animals containing random mixtures of dauer and non-dauer cells. It is possible that the INS-7 positive feedback loop contributes to this cellular conformity. In this model, a downward or upward fluctuation in the level of INS-7 would be amplified, which in turn would bias all of the cells in the animals towards dauer or adult development, respectively.

Additional Downstream Signaling Molecules

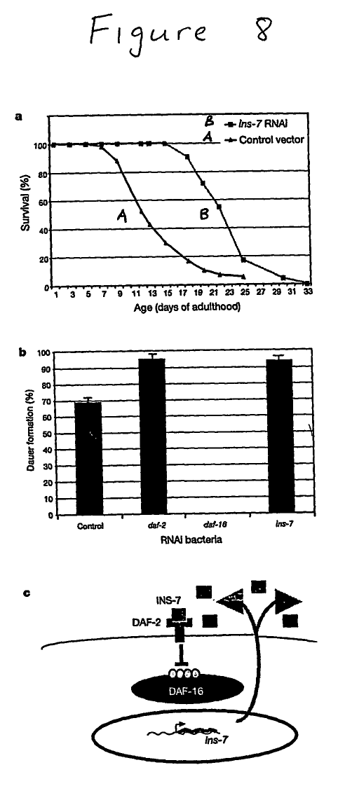

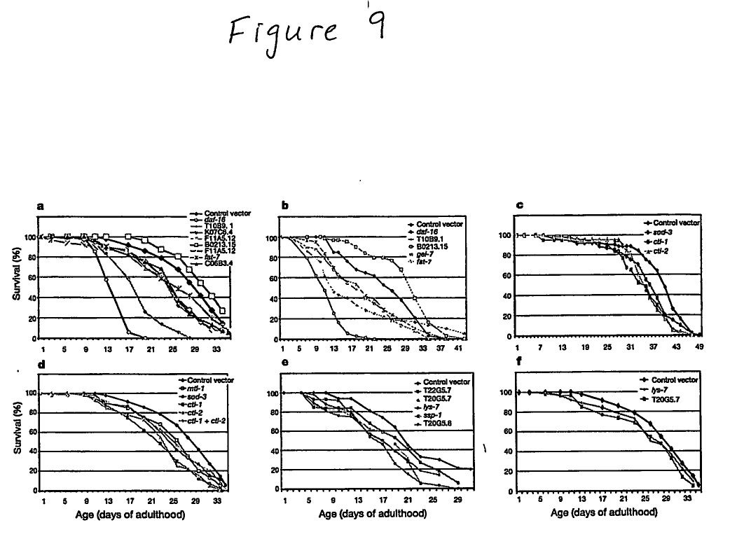

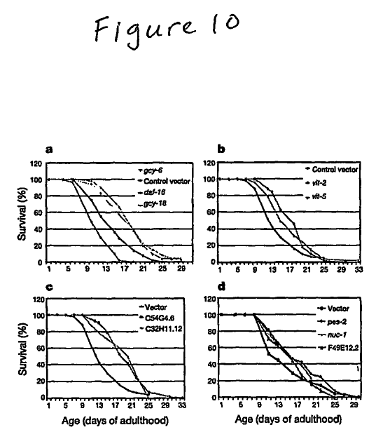

In addition to ins-7, a number of other genes that encoded potential signaling molecules were regulated by DAF-2 and DAF-16. One was a known daf-2/daf-16-regulated gene, scl-1, which encodes a putative secreted protein that promotes longevity (Ookuma et al., Curr. Biol., 13:427-431 (2003)). Furthermore, a large number of class 1 (daf-2 (−)-induced) genes encoded proteins that might potentially participate in the synthesis of a steroid or lipid-soluble hormone, including four cytochrome P450s, two estradiol-17-β-dehydrogenases, two alcohol/short-chain dehydrogenases, several esterases, two UDP-glucuronosyltransferases, and severalfat genes known to function in fatty acid desaturation (Table 7). We investigated the functions of many of these metabolic, steroid, and lipid synthesis genes and found that reducing their activities with RNAi (in daf-2(mu150) mutants) shortened lifespan up to 20% (FIG. 9a, b). Together these findings suggest that the DAF-2 pathway may regulate multiple downstream signaling molecules. We also found that gcy-6 and gcy-18, two receptor guanylate cyclases that are expressed in neurons (Yu et al., Proc. Natl Acad. Sci. USA, 94:3384-3387 (1997)), were repressed under daf-2 (−) conditions (class 2). Inhibiting their activities lengthened the lifespan of daf-2 (+) animals (FIG. 10a). Thus insulin/IGF-1 signaling may affect the animal's response to the environment.

A Broad-based Defense Against Cellular Damage Increases Longevity

A major goal of this study was to identify genes whose products directly influence ageing. We identified two prominent groups of functionally related genes that were candidates for direct effectors. The first group contained a wide variety of stress-response genes. In addition to mtl-1 and sod-3, we found that expression of the catalase genes ctl-1 and ctl-2, the glutathione-S-transferase gene gst-4, and the small heat-shock protein genes were all increased in animals with reduced daf-2 activity and decreased in animals with reduced daf-16 activity. We inhibited the activities of these genes with RNAi, and found that, in each case, the lifespans of daf-2 mutants were shortened, generally between 10-20% (FIG. 9c, d and Table 5). Oxidative stress genes were also tested in daf-2(mu150) mutants (FIG. 9c, d). Because DAF-16 also functions in the wild type to extend lifespan, inhibiting these genes would be predicted to shorten wild-type lifespan as well. This was often the case, although the magnitude of the effect was smaller than in daf-2 (2) mutants (Table 8). Thus, each of these genes functions to promote longevity, probably by preventing or repairing oxidative and other forms of macromolecular damage.

An Antimicrobial Response Lengthens Lifespan

The second prominent set of potential lifespan effectors encoded antimicrobial proteins. Caenorhabditis elegans feeds on bacteria, and, at least under laboratory conditions, wild-type animals exhibit pharyngeal and intestinal bacterial packing as they age (Garigan et al., Genetics, 161:1101-1112 (2002)), and are ultimately killed by proliferating bacteria (Garigan et al., Genetics, 161:1101-1112 (2002)). daf-2 mutants display reduced bacterial pacldng when compared with wild-type nematodes of the same age (Garigan et al., Genetics, 161:1101-1112 (2002)). We found that several genes encoding antibacterial lysosymes were induced in daf-2 mutants, including two intestinally expressed genes, lys-7 (C02A12.4) and lys-8 (C17G10.5), which are also induced when C. elegans is infected with pathogenic Serratia marcescens (Mallo et al., Curr. Biol., 12:1209-1214 (2002)). The saposin-like gene spp-1 (T07C4.4), which has demonstrated antibacterial activity(Banyai et al., Biochim. Biophys. Acta, 1429:259-264 (1998)), was also upregulated in daf-2 (−) animals. To test whether expression of these genes contributes to the longevity of daf-2 mutants, we reduced the activities of several using RNAi. We found that these treatment shortened lifespan of daf-2 mutants (FIG. 9e, f), indicating that these genes contribute to longevity. Antimicrobial genes were also tested in daf-2(mu150) and daf-2(e1370) mutants (FIG. 9e, f).

Other daf-2/daf-16-regulated Genes Also Influence Lifespan

We found a number of other daf-2/daf-16-regulated genes with substantial effects on lifespan. For example, the vitellogenin (yolk protein/apolipoprotein-like) genes vit-2 and vit-5 were downregulated in daf-2 (−) animals and upregulated in daf-16 (−) animals, and we found that reducing their activities lengthened the lifespan of daf-2 (+); ffr-3(pk1426) animals (FIG. 10b). Several proteases and metabolic genes were also class 2 genes, including an aminopeptidase, a carboxypeptidase, an amino-oxidase, an aminoacylase, and pep-2, an oligopeptide transporter, as well as several F-box/cullin/Skp proteins (including skr-8, skr-9 andpes-2) associated with ubiquitin-mediated protein degradation. Inhibition of several of these genes extended the lifespan of daf-2 (+) animals (Table 6). This suggests that daf-2 lifespan extension may involve turnover of specific proteins or metabolites. The glyoxylate cycle gene gei-7 encoding isocitrate lyase/malate synthase, which is upregulated in dauers (Wang et al., Development, 130:1621-1634 (2003)) and hibernating mammals (Davis et al., Biochim. Biophys. Acta, 1051:276-278 (1990)), was upregulated in daf-2 (−) animals. Inhibiting the function of this gene shortened the lifespan of daf-2 (−) mutants substantially, while shortening wild type lifespan only slightly. Thus this alternative metabolic pathway contributes to longevity. A large class of unknown genes containing a shared domain of unknown function (the DUF141 domain) was downregulated in daf-2 mutants, and RNAi of these genes extended lifespan (FIG. 10c). One gene that is repressed in daf-2 mutants and induced in daf-16 mutants, C54G4.6, had a relatively large effect on lifespan (FIG. 10c). This gene shares homology with bacterial orfE/MAF inhibitor of septum formation proteins and with a human protein, ASMTL (Ried et al., Mol. Genet., 7:1771-1778 (1998)). Finally, several other class 2 genes that significantly extended lifespan shared no homology with known genes (FIG. 10d and Table 6; see also Table 8).

A New DNA Sequence in Regulatory Regions of Downstream Genes

To identify potential transcription-factor binding sites, we searched in an unbiased way for common sequence patterns in the upstream regulatory regions of genes in each cluster using two different algorithms. We used the ‘Mobydick’ algorithm (Bussemaker et al., Proc. Natl Acad. Sci. USA, 97:10096-10100 (2000)) to identify short sequences (words) whose statistical distribution suggests that they are meaningful informational units. In this analysis we used sequences taken from a 1-kilobase (kb) region upstream of each gene in the cluster; words that are over-represented in the cluster are candidate transcription-factor binding sites. We also used another algorithm that searches exhaustively for oligonucleotides overrepresented in each cluster (van Helden et al., J Mol. Biol., 281:827-842 (1998)). We found that the sequence T(G/A)TTTAC, which has been shown to be bound by DAF-16 in vitro (Furuyama et al., Biochem. J., 349:629-634 (2000)), was over-represented, suggesting that our set of genes includes many direct DAF-16 targets. Notably, this canonical site was present not only in the promoters of class 1 (daf-2-induced) genes, but also in the promoters of class 2 (daf-2-downregulated) genes (Tables 1 and 2). Thus, DAF-16 may both directly repress and activate gene expression. We also found that a new sequence, CTTATCA, scored highly in both algorithms. Both sequences were present in various combinations in the promoters of both the class 1 and class 2 genes (Tables 5 and 6). The existence of this new site suggests that DAF-16 may regulate its target genes in combination with an additional, as yet unidentified, factor.

Mechanisms That Modulate the Rate of Ageing

Together these findings suggest that the regulation of ageing by the insulin/IGF-I pathway is achieved through a combination of global regulators, such as INS-7 and neuronal signaling components, and a wide variety of genes whose products may affect the ageing process directly. Several DAF-16 target genes that had significant effects on lifespan encoded new proteins, and it will be interesting to learn whether these genes act in unexpected ways to influence lifespan. In addition, many DAF-16 target genes encoded proteins predicted to protect cells from oxidative and other forms of stress. Thus our study provides strong support for the theory that genes that increase resistance to environmental stress contribute to longevity.

In addition, our findings revealed that the ability to ward off microbial infections contributes to the longevity of C. elegans, and that this ability is regulated by insulin/IGF-I signaling. Bacterial infections are a major cause of disease and death in elderly humans. Thus, it will be interesting to learn whether the human insulin or IGF-I systems regulate the susceptibility to bacterial infections by controlling the expression of antimicrobial genes.

It was particularly interesting to find that no single RNAi treatment, other than daf-16 RNAi itself, completely suppressed the lifespan extension of daf-2 mutants. This was true also when we used a mutant strain with increased RNAi sensitivity (Sijen et al., Cell, 107:465-476 (2001)) (Tables 5 and 6; see also Table 8(m)). This result indicates that multiple effector genes, whose expression is coordinated by the DAF-2 pathway, probably act in a cumulative manner to influence ageing. Because by themselves most genes have a relatively small effect on lifespan, it would have been difficult to identify any particular one in a standard genetic screen. Thus this study demonstrates the power of functional microarray analysis for dissecting complex regulatory systems.

Longevity must have evolved not just once, but many times. Insect lifespans range from a few weeks to several years, and those of mammals (and also birds) range from a few years to a century. Evolutionary theory postulates that lifespan is determined by the additive effects of many genes (Kirkwood et al., Nature, 408:233-238 (2000)), consistent with our findings. The beauty of the insulin IGF-I system is that it provides a way to regulate all of these genes coordinately. As a consequence, changes in regulatory genes encoding insulin/lGF-I pathway members or DAF-16 homologues could, in principle, allow changes in longevity to occur rapidly during evolution. Additional evolutionary flexibility may arise from the fact that the insulin and IGF-I system regulates longevity, reproduction, states of diapause and body size independently of one another (Holzenberger et al., Nature, 421:182-187 (2003); Gems et al., Genetics, 150:129-155 (1998); Dillin et al., Science, 298:830-834 (2002); Wolkow et al., Science, 290:147-150 (2000); Clancy et al., Science, 292:104-106 (2001)). Thus, regulatory mutations that affect these traits differentially may allow evolving species to move into environmental niches that favor highly specific life history strategies.

Two studies of genes regulated by daf-16 have been published (Lee et al., Science, 300:644-647 (2003); McElwee et al., Aging Cell, 1:111-121 (2003)). We note that the gene called ins-7 may in fact be referred to by some as ins-30, which corresponds to the gene number cited in that report, ZC334.2.

Methods

Microarray Construction

We used Research Genetics ‘GenePairs’ primers for 18,455 predicted genes to amplify fragments by PCR from C. elegans N2 genomic DNA. PCR products were ethanol precipitated and size-confirmed before printing onto polylysine slides (DeRisi et al., Science, 278:680-686 (1997)).

Strains

Mutations used in this study were: LG1, daf-16 (mu86); LGII, age-1 (hx546), fer-15 (b26), rrf-3 (k426) (Sijen et al., Cell, 107:465-76 (2001)); LGIII, daf-2 (mu150) (Garigan et al., Genetics, 161:1101-1112 (2002)), daf-2 (e1368), daf-2 (e1370); LGIV, fem-1 (hc17); DAF-16::GFP strain, (muEx 110 (pKL99-2 (daf-16::gfp /daf-16b (−))+RF4(rol-6)); daf-16 (mu86) I; daf-2 (e1370) III) (Lin et al., Nature Genet., 28:139-145 (2001)).

RNAi

Bacterial feeding RNAi experiments were carried out as described previously (Dillin et al., Science, 298:830-834 (2002); Fraser et al., Nature, 408:325-330 (2000)). We verified each clone from the RNAi library (Fraser et al., Nature, 408:325-330 (2000)) by PCR and sequence analysis.

Caenorhabdids elegans Growth and Collection

A total of 30,000-50,000 nematodes were collected for each microarray sample. daf-2 and age-1 mutants were synchronized by hypochlorite treatment and L1 arrest, then grown to adulthood on 150 mmNG OP50 plates at 20° C. or 25° C. Synchronized fer-15 (b26);fem-1 (hc17) animals were grown on RNAi bacteria at 25° C. and collected at the indicated time points (FIG. 1a); isopropyl-β-thiogalactoside was added on day 1 of adulthood and RNAi bacteria was supplemented as necessary. Nematodes were washed in M9, dissolved in Trizol (Gibco) and frozen in liquid nitrogen.

Microarray Hybridizations

Standard techniques were used to obtain RNA (Trizol), messenger RNA (Oligotex, Qiagen), complementary DNA (reverse transcription) and Cy-dye-labeled cDNA (DeRisi. et al., FEBS Lett., 470:156-160 (2000)); arrays were hybridized for 18 h at 63° C., washed and scanned. One-half of each time course sample was added to a pool, and every Cy5-labelled sample was compared to this Cy3-labelled mixed reference. Mutant comparisons were done both directly and in a pooled comparison.

Significance Analysis

After array normalization, SAM analysis (Tusher et al., Proc. Natl Acad. Sci. USA, 98:5116-5121 (2001)) was performed on data from nine mutant arrays (one-class response) to identify genes with small but consistent changes. In this set of arrays at a d-value of 1.47, 70 upregulated and 100 downregulated genes were found to be significant (q-value 1/40.0011194) with 0.6207 median false significant genes (Table 7).

Correlation Coefficient Analysis

We calculated a vector comprising the entirety of log ratio comparisons for all the genes with a valid signal at a single time point, to describe each array as a single value. We compared each array in the two time courses to all other arrays in that time course, and the Pearson correlation of the log base-two of these expression ratios was calculated. Five arrays from the set of 60 time points did not correlate with neighboring time points, and were eliminated.

Cluster Analysis

After normalization, log transformation and quality confirmation through correlation coefficient analysis, data from 55 RNAi arrays and 5 mutant arrays were imported into Gene Cluster (Eisen et al., Proc. Natl Acad. Sci. USA, 95:14863-14868 (1998)) for fold-cutoff analysis and hierarchical clustering. Genes were filtered to obtain only those that were present in 80% of the 60 arrays in the data set and which met a max-min of 4-fold, 8-fold or 16-fold criterion. A total of 7,380 genes met a 4-fold cutoff, 2,734 genes met an 8-fold cutoff and 1,280 genes met a 16-fold cutoff over the entire set of 55 RNAi arrays and five mutant arrays. The filtered set was hierarchically clustered, a self-organized map was constructed with 300,000 iterations, and the gene set was displayed in TreeView (Eisen et al., Proc. Natl Acad. Sci. USA, 95:14863-14868 (1998)).

Upstream Sequence Analysis

The sequence 1 kb upstream of the translation start site of each open reading frame was assembled and subjected to two algorithms to search for potential transcription-factor binding sites. Exact repeats of length 14 or longer were removed before building the Mobydick (Bussemaker et al., Proc. Natl Acad. Sci. USA, 97:10096-10100 (2000)) dictionary; words were screened by contrasting the frequency of occurrences in the cluster to that in the upstream regions of all the genes in the genome. We also searched for oligonucleotides over-represented in the cluster (van Helden et al., J. Mol. Biol., 281:827-842 (1998)). The occurrence of the identified sequences in the 5 kb upstream of each gene was then determined (van Helden et al., Yeast, 16:177-187 (2000)).

Survival Analyses

Our lifespan analysis focused on a subset of genes whose expression profiles changed in opposite ways under daf-2 (2) and daf-16 (2) conditions. Genes were prioritized by fold expression change and by interesting gene function. The bacteria for 58 genes (Tables 5 and 6) were selected from the RNAi library (Fraser et al., Nature, 408:325-330 (2000)). A total of 60-70 nematodes were used per experiment as described previously (Kenyon et al., Nature, 366:461-464 (1993); Apfeld et al., Cell, 95:199-210 (1998)). The first day of adulthood was used as t ¼0, and the log-rank (Mantel-Cox) method was used to test the null hypothesis (StatView 5.01, SAS Software). fer-15 (b26);fem-1 (hc17) animals were grown at 25° C. on RNAi bacteria and lifespans were measured at this temperature unless otherwise indicated. daf-2 (mu150) nematodes were measured at 25° C. in one trial, and in subsequent tests were raised at 20° C. then shifted to 25° C. at L3. rrf-3 (pk1426) (Sijen et al., Cell, 107:465-476 (2001)) mutants were treated at 20° C. in one experiment; in subsequent experiments, the nematodes were shifted to 25° C. at L2 through young adulthood to induce sterility, and adult lifespan was measured at 20° C. rif-3 (pk1426); daf-2 (e1370) lifespan tests were done at 20° C.

Dauer Tests

daf-2 (e1370) nematodes were grown on RNAi bacteria at 20° C., F1 eggs were incubated at 22.5° C., and animals were scored for dauer arrest 72 h later.

1. A method for identifying a compound that modulates aging, the method comprising the steps of: (i) contacting the compound with a polypeptide, wherein the polypeptide is encoded by a nucleic acid that hybridizes under stringent conditions to a nucleic acid listed in Tables 1 or 3-7, or a nucleic acid encoding a polypeptide listed in Tables 1 or 3-7or mammalian homologs and orthologs thereof; and (ii) determining the functional effect of the compound upon the polypeptide.

2. The method of claim 1, wherein the homolog or ortholog is a human homolog or ortholog.

3. The method of claim 2, wherein the human homolog or ortholog is a human cellular stress-response gene, a human antimicrobial gene, a human metabolic gene, a human steroid or lipid-soluble hormone synthesis gene, or a human fatty acid desaturation gene.

4. The method of claim 2, wherein the human homolog or ortholog is a cytochrome P450, an estradiol-17-0-dehydrogenase, a alcohol/short-chain dehydrogenase, an esterase, a UDP-glucuronosyltransferase, an aminopeptidase, a carboxypeptidase, an amino-oxidase, an aminoacylase, an oligopeptide transporter, metallothionein, a receptor guanylate cyclase, a mitochondrial superoxide dismutase, a catalase, lysosyme, saposin, vitellogenin, glutathione-S-transferase, heat-shock protein, heat shock factor, an F-box/cullin/Skp protein, an isocitrate lyase, a malate synthase ASMTL, insulin, IFG1 or IFG2.

5. The method of claim 1, wherein the polypeptide is encoded by a nucleic acid that hybridizes under stringent conditions to a nucleic acid listed in Tables 5-6, or a nucleic acid encoding a polypeptide listed in Tables 5-6, or mammalian homologs or orthologs thereof.

6. The method of claim 1, wherein the functional effect is determined in vitro.

7. The method of claim 6, wherein the functional effect is determined by measuring enzymatic activity.

8. The method of claim 6, wherein the functional effect is determined by measuring ligand, substrate, or cofactor binding to the polypeptide.

9. The method of claim 6, wherein the functional effect is determined by measuring interaction between a nucleic acid and the polypeptide.

10. The method of claim 1, wherein the polypeptide is expressed in a eukaryotic host or host cell and the polypeptide is contacted with the compound in a living cell.

11. The method of claim 10, wherein the host cell is derived from C. elegant, mouse, rat, or human.

12. The method of claim 10, wherein the host is C. elegans, mouse, rat, or human.

13. The method of claim 10, wherein the functional effect is a determined by measuring ligand, substrate, or cofactor binding to the polypeptide.

14. The method of claim 10, wherein the functional effect is determined by measuring transcriptional activation.

15. The method of claim 10, wherein the functional effect is determined by evaluating age-associaied parameters.

16. The method of claim 10, wherein the functional effect is determined by evaluating expression of an age-associated gene.

17. The method of claim 15, wherein the age-associated parameter is lifespan.

18. The method of claim 1, wherein the modulation is inhibition of aging.

19. The method of claim 1, wherein the compound is an antibody, an antisense molecule, or a small molecule.

20. The method of claim 18, wherein inhibition of aging occurs by inhibition of a polypeptide encoded by a nucleic acid that hybridizes under stringent conditions to a nucleic acid encoding a polypeptide comprising an amino acid sequence selected from the group consisting of the genes listed in Tables 1 or 3-7 or human homologs and orthologs thereof.

21. A method for evaluating a compound for modulation of aging, the method comprising the steps of: (i) contacting the compound with a polypeptide, wherein the polypeptide is encoded by a nucleic acid that hybridizes under stringent conditions to a nucleic acid listed in Tables 1 or 3-7, or a nucleic acid encoding a polypeptide listed in Tables 1 or 3-7, or mammalian homologs and orthologs thereof (ii) determining the functional effect of the compound upon the polypeptide; and (iii) contacting a host or host cell expressing the protein and evaluating an age-associated parameter of the host or host cell, thereby evaluating a compound for modulation of aging.

22. The method of claim 21, wherein the homolog or orthologs is a human homolog or ortholog.

23. The method of claim 22, wherein the human homolog or ortholog is a human cellular stress-response gene, a human antimicrobial gene, a human metabolic gene, a human steroid or lipid-soluble hormone synthesis gene, or a human fatty acid desaturation gene.

24. The method of claim 22, wherein the human homolog or ortholog is a cytochrome P450, an estradiol-17-β-dehydrogenase, a alcoholshort-chain dehydrogenase, an esterase, a UDP-glucuronosyltransferase, an aminopeptidase, a carboxypeptidase, an amino-oxidase, an aminoacylase, an oligopeptide transporter, metallothionein, a receptor guanylate cyclase, a mitochondrial superoxide dismutase, a catalase, lysosyme, saposin, vitellogenin, glutathione-S-transfdrase, heat-shock protein, an F-box/cullin/Skp protein, an isocitrate lyase, a malate synthase ASMTL, insulin, IFG1 or IFG2.

25. The method of claim 21, wherein the polypeptide is encoded by a nucleic acid that hybridizes under stringent conditions to a nucleic acid listed in Tables 5-6, or a nucleic acid encoding a polypeptide listed in Tables 5-6, or mammalian homologs and orthologs thereof.

26. The method of claim 21, wherein the polypeptide is recombinant.

27. The method of claim 21, wherein the compound is an antibody, an antisense molecule, or a small molecule.

28. The method of claim 21, wherein the functional effect is a physical effect.

29. The method of claim 21, wherein the functional effect is a chemical effect.

30. The method of claim 21, wherein the functional effect is a phenotypic effect.

31. The method of claim 21, wherein the functional effect is determined in vitro.

32. The method of claim 21, wherein the functional effect is determined in a eukaryotic host organism or host cell.

33. The method of claim 21, wherein the age-associated parameter is lifespan, wherein the age-associated parameter is stress resistance.

34. A compound that modulates an aging process, wherein the compound is identified by the method of claim 1.

35. The compound of claim 34, wherein the compound is an antibody, an antisense molecule, or a small molecule.

36. A method of modulating lifespan regulation in a subject, the method comprising the step of administering to the subject an effective amount of a compound identified using the method of claim 1.

37. The method of claim 36, wherein the subject is an adult.

38. The method of claim 37, wherein the subject is a non-diabetic, non-obese adult.

39. The method of claim 37, wherein the subject is not at risk for or does not have a premature aging disorder.

40. The method of claim 37, wherein the subject is a healthy adult.

41. A method of increasing lifespan or treating premature aging in a subject, the method comprising the step of administering to the subject an effective amount of a compound identified using the method of claim 1.

42. The method of claim 41, wherein the aging process is abnormal.

43. The method of claim 42, wherein the abnormal aging process is selected from Werner syndrome, Hutchinson-Guilford disease, Bloom's syndrome, Cockayne's syndrome, ataxia telangiectasia, and Down's syndrome.

44. The method of claim 41, wherein the aging process is normal.

45. The method of claim 41, further comprising the step of evaluating an age-associated parameter of the subject.

46. A method of identifying a compound that modulates aging, the method comprising the steps of: (i) contacting a test compound to a living or biochemical system that comprising a C. elegans target protein selected from the group consisting of: a protein in Tables 1 or 3-7; and (ii) evaluating a property associated with the target protein; and (iii) evaluating an aging-associated parameter of a C. elegans organism contacted with the test compound.

47. A C. elegans nematode that (1) has a deficiency in at least some cells for an endogenous activity, the deficiency generated by dsRNA in the cells, and (2) has an average lifespan of at least 40% greater than an otherwise identical nematode without the deficiency.

48. A method of identifying a gene or gene product that modulates aging, the method comprising the steps of: (i) providing the nematode of claim 47; (ii) introducing a heterologous gene that encodes a heterologous polypeptide into the nematode; (iii) expressing the heterologous gene in the nematode or a progeny of the nematode under conditions wherein the heterologous polypeptide is produced; and (iv) monitoring an age-associated parameter of the nematode or the progeny of the nematode.

49. The method of claim 48, further comprising contacting a test compound to the nematode or the progeny prior to or during the monitoring.

50. A method of evaluating a plurality of compounds, the method comprising the steps of: providing a plurality of compounds; for each compound of the plurality, evaluating a functional effect of the respective compound on a polypeptide that is encoded by a nucleic acid that hybridizes under stringent conditions to a nucleic acid listed in Tables 1 or 3-7, or a nucleic acid encoding a polypeptide listed in Tables 1 or 3-7 or mammalian homologs and orthologs thereof; and if the compound has a functional effect as determined by a criterion, contacting the compound to a cell or organism, and evaluating an-age related parameter of the cell or organism.

51. The method of claim 50 wherein the criterion is a preselected value.

52. The method of claim 50, wherein the criterion is a preselected statistical significance.

53. The method of claim 50, wherein the plurality of compounds comprises a library of structurally related chemical compounds.

54. A method of altering lifespan regulation in a cell or organism, the method comprising: increasing expression of at least two class 1 genes in the cell or organism.

55. The method of claim 54 wherein the step of increasing comprises introducing one or more heterologous nucleic acid that encode the at least two class 1 genes.

56. The method of claim 54, wherein the step of introducing comprises introducing a single nucleic acid that comprises coding sequences for the at least two class 1 genes.

57. A compound that modulates an aging process, wherein the compound is identified by the method of claim 21.

58. A method of modulating lifespan regulation in a subject, the method comprising the step of administering to the subject an effective amount of a compound identified using the method of claim 21.

59. The method of claim 37, wherein the subject is a healthy adult 41. A method of increasing lifespan or treating premature aging in a subject, the method comprising the step of administering to the subject an effective amount of a compound identified using the method of claim 21.
